(19) United States
(12) Patent Application Publication
(10) Pub. No.: US 2010/0272299 A1
(43) Pub. Date: Oct. 28, 2010

(54) BODY-WORN WIRELESS TRANSDUCER MODULE

(76) Inventors: Koenraad Van Schuylenbergh, Niel (BE); Nicolaas Van Ruiten, Niel (BE); Luc Van Immerseel, Niel (BE)

Correspondence Address: TRASKBRITT, P.C., P.O. BOX 2550, SALT LAKE CITY, UT, 84110, US

(21) Appl. No.: 12/734,308

(22) PCT Filed: Oct. 30, 2007
(86) PCT No.: PCT/EP2007/061691

§ 371 Date: Apr. 22, 2010

(51) Int. Cl. H04R 25/00; A61F 2/18

(52) U.S. Cl. 381/315; 623/10

(57) ABSTRACT

The present invention relates to an externally worn transducer module (1) for use with an implant (101), which module (1) comprises: —a transducer (5) for converting energy into electrical signals, —a wireless interface (19), configured to transfer data signals to and/or from the implant (101), and receive electrical power from the implant (101), —circuitry (8) operably connected to the transducer (5) and interface (19) configured to: —receive electrical power from the interface (19), —convert electrical signals generated by the transducer into data signals responsive to the electrical signals, and —provide data signals to the interface (19), and —housing (2) that forms a protective body of the module (1), which housing (1) is configured for external attachment to the body of the wearer. It particularly relates to a microphone module for use with a hearing aid implant. It also relates to a kit comprising the module and optionally comprising the implant, and tools for user insertion and maintenance.

The present invention relates to a body worn wireless transducer module which detects energy (e.g. sound, heat, light (images, intensity, colour), vibration, compression or tensile energy), converts it into electrical signals, and wirelessly exchanges data signals responsive to the electrical signals to an implant in the body. It is particularly applicable to hearing aids where the transducer is a microphone, though it may equally be used with other transducers such as heat, light, vibration as mentioned elsewhere herein.

The current trend in hearing-aid implants is towards fully implanted solutions, whereby both the microphone and audio processing unit are surgically implanted. This provides an advantage of discrete wearing and makes the hearing aid less prone to damage or detachment by shock movements. Typically, the implanted microphone is placed in either the middle ear cavity, against the skull underneath the skin, or underneath the ear-canal skin.

While users of fully implanted hearing aids enjoy the benefits of discretion and shock resistance, the improvement to hearing is mixed. The typical problems associated with hearing aids with an implanted microphone include:
poor signal-to-noise performance from skull-based microphones, owing to the layer of skin and soft tissue that severely attenuates sound. Subcutaneous sound pressure level (SPL) is typically 30 dB below the level above the skin;
unwanted body noises picked up by microphones mounted against the skull e.g. the sound of muscle tissue against the microphone, and chewing and biting sounds that conduct through the skull and also vibrate the microphone;
Larsen feedback with the actuator, or with the oval and round windows in cases where the microphone is implanted in the middle-ear cavity;
movement of microphones underneath the ear-canal skin. Microphones implanted in the soft tissue around the outer ear canal are not well accepted by the human body; they tend to migrate and cause tissue reaction. The region of the ear canal is also subjected to substantial jaw movements during eating, talking, yawning etc which causes additional movement and dislodgement.

Implanted hearing aids are known in the art. For example, U.S. Pat. No. 3,764,748 discloses a battery-powered hearing aid positioned within the external ear canal that picks up auditory signals from the eardrum. The hearing aid subsequently amplifies and/or transmits such signals directly to appropriate sound receiving mechanisms, natural or solid-state or both, located on the oval window, the round window, or the promontory leading into the inner ear. U.S. Pat. No. 4,800,884 describes a magnetic induction hearing aid where the microphone, the amplifying electronics, the battery and a coil are contained in a single housing which is located deep in the ear canal. A magnet is attached to portions of the middle ear by means of a malleus clip or by implantation between the tympanic membrane and the malleus. The magnet is vibrated by interaction with the magnetic field produced by the coil. The ear-canal unit can also be used in conjunction with the ossicular replacement prosthesis described in U.S. Pat. No. 4,817,607, that also contains a magnet in the head of the prosthesis. U.S. Pat. Nos. 4,957,478 and 5,015,224 disclose a partially implantable hearing aid based on magnetic induction. Its outer ear-canal unit contains a power source, i.e. battery. U.S. Pat. No. 5,015,225 teach a bone-conduction hearing aid with a battery-powered microphone unit in the external ear canal. U.S. Pat. No. 6,010,532 describes a binaural set up in, but without ear-canal microphones.

Other prior art includes US 2006/0215863 describing a protective barrier for a microphone; US 2006/0147071, U.S. Pat. No. 7,013,016, U.S. Pat. No. 6,795,562, U.S. Pat. No. 6,164,409, U.S. Pat. No. 5,970,157, U.S. Pat. No. 5,712,918, U.S. Pat. No. 5,401,920, U.S. Pat. No. 5,327,500, U.S. Pat. No. 5,278,360, and U.S. Pat. No. 4,553,627, all describing wax barriers; US 20070003087 describing the use of a hydrophobic barrier to protect an ear-canal microphone; US 2005/0249370 describing a tool to remove an in-the-ear hearing aid that is inserted deep into the ear canal; US 2005/0018867 describing a tool to install and remove an ear wax guard; U.S. Pat. No. 7,127,790 describing a method to install and remove an ear wax guard; US 2005/0018866 describes another ear wax barrier and also shows dome tips with multiple dome flanges.

It is an aim of the present invention to provide a new configuration for an implanted hearing aid, that has the advantages of discrete wearing of implantable hearing aids discussed here, but which has improved sound performance, shock resistance and convenience.

### FIGURE LEGENDS

FIG. 1 Shows an illustration of a microphone module 1 of the invention in combination with a fully implantable cochlear implant, showing the battery-powered implant control unit 15, connected to a cochlear electrode 17, and the implanted interface 16.

FIGS. 2a and 2b. Illustration of an embodiment of a microphone module of the invention with single buffering structure, and a sound port facing the tympanic membrane end of the housing. Perspective (FIG. 2a) and longitudinal cross section (FIG. 2b) views are shown.

FIGS. 2c and 2d. Perspective (FIG. 2c) and longitudinal cross section (FIG. 2d) views of an embodiment of a microphone module of the invention marked with dimension indications.

FIGS. 3a and 3b. Illustration of an embodiment of a microphone module of the invention with two buffering structure, and a sound port facing the tympanic membrane end of the housing. Perspective (FIG. 3a) and longitudinal cross section (FIG. 3b) views are shown.

FIG. 4a. Illustration of an embodiment of a microphone module cross-section with two buffering structure, and a sound port facing the pinna end of the housing.

FIG. 4b. Illustration of an embodiment of a microphone module cross-section with two buffering structure, and a sound port on the longitudinal body of the housing.

FIG. 5. Illustration of an embodiment of a microphone module of the invention with two buffering structures, and a sound port facing the tympanic membrane end of the housing, where the buffering structure is provided with a perforation.

FIG. 6. Illustration of an embodiment of a microphone module of the invention with two buffering structures, and a sound port facing the tympanic membrane end of the housing, where the buffering structures are provided with perforations that extends to the extremity of the buffering structures.

FIGS. 7 and 8. Illustration of a replaceable membrane of the invention shown in perspective view (FIG. 7) and in cross-section (FIG. 8).

FIG. 9 Perspective view of a placement tool for insertion and removal of a replaceable membrane.

FIG. 10. Cross-section through a module of the invention in situ showing inductive module and implant coils in a parallel, coaxial alignment. The dashed lines represent the magnetic field generated by the implanted coil.

FIG. 11. Cross-section through a module of the invention in situ showing inductive module and implanted coil in a perpendicular alignment. The dashed lines represent the magnetic field generated by the implanted coil.

FIG. 12 Perspective view of an outer ear canal, provided with an implant interface comprising orthogonal coils having currents out of phase by 90° capable of generating a rotating magnetic field.

FIG. 12 A View along the outer canal of the location of two orthogonal having currents out of phase by 90° capable of generating a rotating magnetic field.

FIG. 13A shows a plot of the current (I) as a function of time (t) of two orthogonal coils having AC currents out of phase by 90°.

FIG. 13B shows the net resulting magnetic field from the currents provided in FIG. 13A.

FIGS. 14 and 15. Illustration of a microphone module where interface induction coils are embedded in the buffering structures. Perspective (FIG. 15) and longitudinal cross section (FIG. 14) views are shown.

FIG. 16. Skull side view illustrating the close proximity of the mandibular joint to the ear canal.

FIG. 17. Vertical cross-section through the human hearing system indicating locations for an implant interface.

FIG. 18. Horizontal cross-section through the human hearing system indicating another location for an implant interface.

FIG. 19. Shows the module of the invention in situ, where module is wirelessly connected to the implant with a conductive or capacitive coupling.

FIG. 20. Illustrates alternative module locations with an inductive coupling to the implant.

FIG. 21. Illustrates possible locations for a module worn on the outer ear.

FIG. 22. Module located on the outer ear, as seen from the dorsal view of the patient.

FIG. 23. Illustrates a microphone module placed in an opening that extends through the pinna to form a passage, whereby the implant and module interface coils are in a coaxial alignment.

FIG. 24. Illustrates a microphone module placed in an opening that extends through the pinna to form a passage, whereby the implant and module interface coils are in an overlapping alignment.

FIG. 25. Illustrates a microphone module placed in an opening that extends partially through the pinna to form a cavity, whereby the implant and module interface coils are in a coaxial alignment. The microphone module is held in place by a knob-like protrusion.

FIG. 26. Illustrates a microphone module placed in an opening that extends partially through the pinna to form a cavity, whereby the implant and module interface coils are in an overlapping alignment. The microphone module is held in place by a knob-like protrusion.

FIG. 27. Illustrates a microphone module placed in an opening that extends partially through the pinna to form a cavity, whereby the implant and module interface coils are in a coaxial alignment. The microphone module is held in place by magnets.

FIG. 28. Example of a microphone module placed against the skin, whereby the implant and module interface coils are in an overlapping alignment.

FIG. 29. Wiring configuration of a module and implant of the invention.

FIG. 30 Illustrates a dual microphone configuration with a single hearing aid capturing audio information from microphone modules in both ears and driving actuators in both ears.

FIG. 31 Illustrates a dual microphone configuration with two autonomous hearing aids exchanging co-coordinating data via a mutual link.

FIG. 32 Illustrates a dual microphone configuration comprising a ‘master’ hearing aid on the left side and a ‘slave’ unit on the right connected via a mutual link. The ‘master’ implant also powers the ‘slave’ in this specific example.

### SUMMARY OF SOME EMBODIMENTS OF THE INVENTION

One embodiment of the invention is an externally worn transducer module (1) for use with an implant (101), which module (1) comprises:
a transducer (5) for converting energy into electrical signals,
a wireless interface (19), configured to transfer data signals to and/or from the implant (101), and receive electrical power from the implant (101),
circuitry (8) operably connected to the transducer (5) and interface (19) configured to:
   receive electrical power from the interface (19),
   convert electrical signals generated by the transducer into data signals responsive to the electrical signals, and
   provide data signals to the interface (19), and
a housing (2) that forms a protective body of the module (1), which housing (1) is configured for external attachment to the body of the wearer.

Another embodiment of the invention is a module (1) as described above, wherein the transducer is a microphone transducer (5), and the implant is a hearing implant (101).

Another embodiment of the invention is a module (1) as described above, wherein the housing (1) is configured for insertion into the outer ear canal (12).

Another embodiment of the invention is a module (1) as described above, wherein the housing (1) is configured for attachment to the pinna (10).

Another embodiment of the invention is a module (1) as described above, wherein the wireless interface (19) is configured to receive electrical power inductively, conductively, capacitively or optically.

Another embodiment of the invention is a module (1) as described above, wherein the circuitry (8) comprises a rectifier configured to convert AC voltage received by the wireless interface (19) to DC voltage, to provide said electrical power.

Another embodiment of the invention is a module (1) as described above, wherein the circuitry (8) is configured to modulate the electric power consumption of the circuitry (8) responsive to the data signals, thereby transferring data signals via the interface (19) to the implant (101).

Another embodiment of the invention is a module (1) as described above, wherein the circuitry (8) is configured to modulate the tuning frequency of the interface (19) responsive to the data signals, thereby transferring data signals via the interface (19) to the implant (101).

Another embodiment of the invention is a module (1) as described above, wherein the circuitry (8) is further configured to detect variations in voltage of electrical power received by the interface (19) from the implant (101), which variations correspond to data signals sent by the implant (101).

Another embodiment of the invention is a module (1) as described above, wherein the circuitry (8) is further configured to detect variations in frequency of electrical power received by the interface (19) from the implant (101), which variations correspond to data signals sent by the implant (101).

Another embodiment of the invention is a module (1) as described above, wherein the circuitry (8) is further configured to detect variations in phase of electrical power received by the interface (19) from the implant (101), which variations correspond to data signals sent by the implant (101).

Another embodiment of the invention is a module (1) as described above, whereby the data signals are amplitude modulated signals, frequency modulated signals, phase modulated signals, pulse width modulated signals, pulse sequences, pulse sequences with an SPL (sound pressure level)-depending frequency, pulse sequences with an SPL-depending pulse width, pulse sequences with an SPL-depending pulse phase, or a digitally encoded pulse sequences.

Another embodiment of the invention is a module (1) as described above, whereby the circuitry (8) is configured to process the audio signal generated by the microphone transducer (5) prior to conversion into data signals.

Another embodiment of the invention is a module (1) as described above, wherein said dynamic processing is compression or expansion.

Another embodiment of the invention is a module (1) as described above, whereby the circuitry is configured to filter the audio signal generated by the microphone transducer (5) prior to conversion into data signals.

Another embodiment of the invention is a module (1) as described above, whereby said filtering is low-pass, high-pass, or band-pass filtering.

Another embodiment of the invention is a module (1) as described above, whereby the circuitry is configured to process the audio signal generated by the microphone transducer (5) to provide noise cancellation, frequency shifts, or suppression of Larsen feedback prior to conversion into data signals.

Another embodiment of the invention is a module (1) as described above, wherein the wireless interface (19) configured to receive energy through magnetic induction, comprises one or more inductive coils.

Another embodiment of the invention is a module (1) as described above, wherein the wireless interface (19) configured to receive energy through electrical conduction, comprises one or more contact electrodes.

Another embodiment of the invention is a module (1) as described above, wherein the wireless interface (19) configured to receive energy through capacitive coupling, comprises one or more capacitors.

Another embodiment of the invention is a module (1) as described above, wherein the wireless interface (19) configured to receive energy through optical coupling, comprises one or more photovoltaic cells.

Another embodiment of the invention is a module (1) as described above, wherein the wireless interface (19) further comprises a light source, and wherein the circuitry (8) is configured to modulate the output of the light source responsive to the data signals, thereby transferring data signals to the implant (101).

Another embodiment of the invention is a module (1) as described above, wherein said light source is an infrared or visible light LED.

Another embodiment of the invention is a module (1) as described above, further comprising a telecoil, operably connected to the circuitry (8).

Another embodiment of the invention is a module (1) as described above, further comprising a light sensor, preferably an infrared sensor, operably connected to the circuitry (8).

Another embodiment of the invention is a module (1) as described above, further comprising a radio receiver operably connected to the circuitry (8).

Another embodiment of the invention is a module (1) as described above, where the housing comprises a sound port (4) configured to channel sound energy to the microphone transducer (5).

Another embodiment of the invention is a module (1) as described above, where the sound port (4) comprises a means to receive a protective membrane.

Another embodiment of the invention is a module (1) as described above, where the sound port (4) is covered by a protective membrane.

Another embodiment of the invention is a module (1) as described above, wherein said protective membrane is replaceable.

Another embodiment of the invention is a module (1) as described above, wherein said protective membrane is liquid impermeable, and gas or vapour permeable.

Another embodiment of the invention is a module (1) as described above, wherein said protective membrane comprises Gore-tex.

Another embodiment of the invention is a module (1) as described above, wherein said protective membrane comprises Gore-Tex XCR, eVent breathable fabric or Entrant breathable fabric.

Another embodiment of the invention is a module (1) as described above, further comprising a withdrawal cord or pin.

Another embodiment of the invention is a module (1) as described above, wherein at least part of the outer surface of the housing (2) is radially extended with one or more buffering structures (3, 3′), configured to bridge a gap between the inner wall of the outer ear canal (12) and the outer surface of the housing (2) in situ.

Another embodiment of the invention is a module (1) as described above, wherein a buffering structure (3, 3′) is an annular ring, attached to the housing (2) towards a tympanic membrane end (7), and circumferentially extending outwards and backwards towards of the pinna end (6) of the housing (2), so creating a conical flap.

Another embodiment of the invention is a module (1) as described above, wherein the extremity (28) of the buffering structure (3, 3′) describes a circle.

Another embodiment of the invention is a module (1) as described above, wherein the extremity (28) of the buffering structure (3, 3′) describes an oval.

Another embodiment of the invention is a module (1) as described above, wherein a buffering structure (3, 3′) is provided with one or more perforations (20) configured to allow the passage of sound waves therethrough.

Another embodiment of the invention is a module (1) as described above, wherein a buffering structure (3, 3′) is provided with one or more notches (18) disposed towards the extremity (28) of the buffering structure (3, 3′).

Another embodiment of the invention is a module (1) as described above, wherein the number of buffering structures (3, 3′) is between 1 and 4.

Another embodiment of the invention is a module (1) as described above, wherein the buffering structure (3, 3′), is made of medical-grade silicone or rubber.

Another embodiment of the invention is a kit comprising a transducer module (1) as defined above.

Another embodiment of the invention is a kit as described above, wherein the transducer is a microphone module.

Another embodiment of the invention is a kit as described above, further comprising an implant having a control unit (15), and adapted to transfer data signals to and/or from the module (1), and to provide electrical power to the module (1) via an implant wireless interface (16) operably connected to the control unit (15).

Another embodiment of the invention is a kit as described above, wherein the implant wireless interface (16) is configured to provide electrical power through magnetic induction, electrical conduction, capacitive coupling or optical coupling.

Another embodiment of the invention is a kit as described above, wherein the implant wireless interface (16) configured to provide electrical power through magnetic induction, comprises one or more inductive coils.

Another embodiment of the invention is a kit as described above, wherein the implant wireless interface (16) configured to provide electrical power through electrical conduction, comprises one or more contact electrodes suitable for implantation below the skin.

Another embodiment of the invention is a kit as described above, wherein the implant wireless interface (16) configured to provide electrical power through capacitive coupling, comprises one or more capacitor plates.

Another embodiment of the invention is a kit as described above, wherein the implant wireless interface (16) configured to provide electrical power through optical coupling, comprises one or more light sources.

Another embodiment of the invention is a kit as described above, comprising a microphone module (1) wherein:
wherein the wireless interface (19) further comprises a light source, and wherein the circuitry (8) is configured to modulate the output of the light source responsive to the data signals, thereby transferring data signals to the implant (101), and
wherein the implant wireless interface (16) further comprises a light sensor for the reception of data signals transferred by the light source of the microphone module wireless interface (19).

Another embodiment of the invention is a kit as described above, further comprising one or more replaceable protective membranes suitable for attachment to a sound port (4) of the module (1).

Another embodiment of the invention is a kit as described above, where said protective membrane is as defined above.

Another embodiment of the invention is a kit as described above, wherein said replaceable protective membrane is a C-barrier.

Another embodiment of the invention is a kit as described above, further comprising a protective membrane placement tool configured to allow attachment and/or removal the replaceable protective membrane from the module (1).

Another embodiment of the invention is a kit as described above, further comprising a microphone module (1) placement tool, configured to allow a user to insert and/or remove the microphone module (1) from the outer ear canal (12).

Another embodiment of the invention is an implant having a control unit (15), and adapted to transfer data signals to and/or from a module (1) according to any of claims 1 to 42, and to provide electrical power to the module (1) via an implant wireless interface (16) operably connected to the control unit (15).

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a sound port” means one sound port or more than one sound port.

The recitation of numerical ranges by endpoints includes all integer numbers and, where appropriate, fractions subsumed within that range (e.g. 1 to 5 can include 1, 2, 3, 4 when referring to, for example, a number of sound ports, and can also include 1.5, 2, 2.75 and 3.80, when referring to, for example, measurements). The recitation of end points also includes the end point values themselves (e.g. from 1.0 to 5.0 includes both 1.0 and 5.0).

The present invention relates to an externally worn transducer module for use with an implantable system, which module comprises a transducer, circuitry for converting electrical signals generated by the transducer into data signals responsive to the electrical signals, which data signal are received by the implant, and a housing which protectively covers the components and forms the body of the module. The module is suitable for concealed insertion on the body of the subject i.e. on or attached to the skin, for example, for inserted into the external ear canal or attached to the pinna. The transducer module is autonomous, meaning it operates without the need for hardwiring to any other device, such as to the implant or an external power source; power is received from and data is exchanged with the implantable system using a wireless link.

The description that follows describes an embodiment of module that is a microphone module. However, the invention is by no means limited thereto; skilled person may adapt the concepts described herein without inventive burden to other transducers that are sensitive to parameters such as heat, light (cameras, light intensity sensors, colour sensors), and vibration as mentioned elsewhere herein.

According to one embodiment, the present invention relates to an externally worn microphone module for use with an implantable hearing system, which module comprises a microphone transducer, circuitry for converting electrical signals generated by the microphone into data signals responsive to the electrical signals, which data signals are received by the implant as sound information, and a housing which protectively covers the components and forms the body of the module. The module is suitable for concealed insertion on the body of the subject i.e. on or attached to the skin, for example, for inserted into the external ear canal or attached to the pinna. The microphone module is autonomous, meaning it operates without the need for hardwiring to any other device, such as to the implant or an external power source.

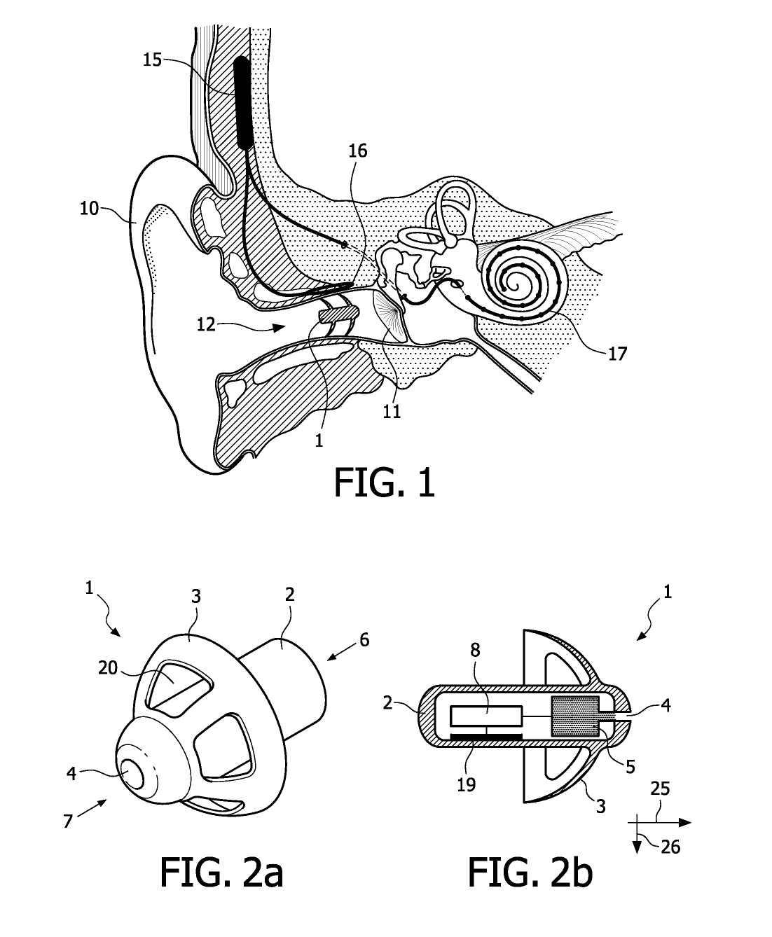

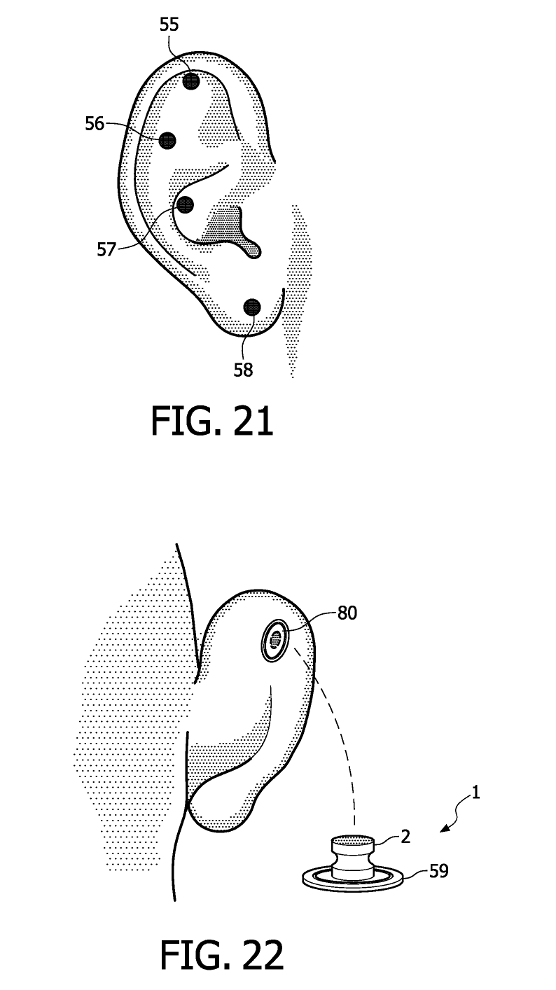

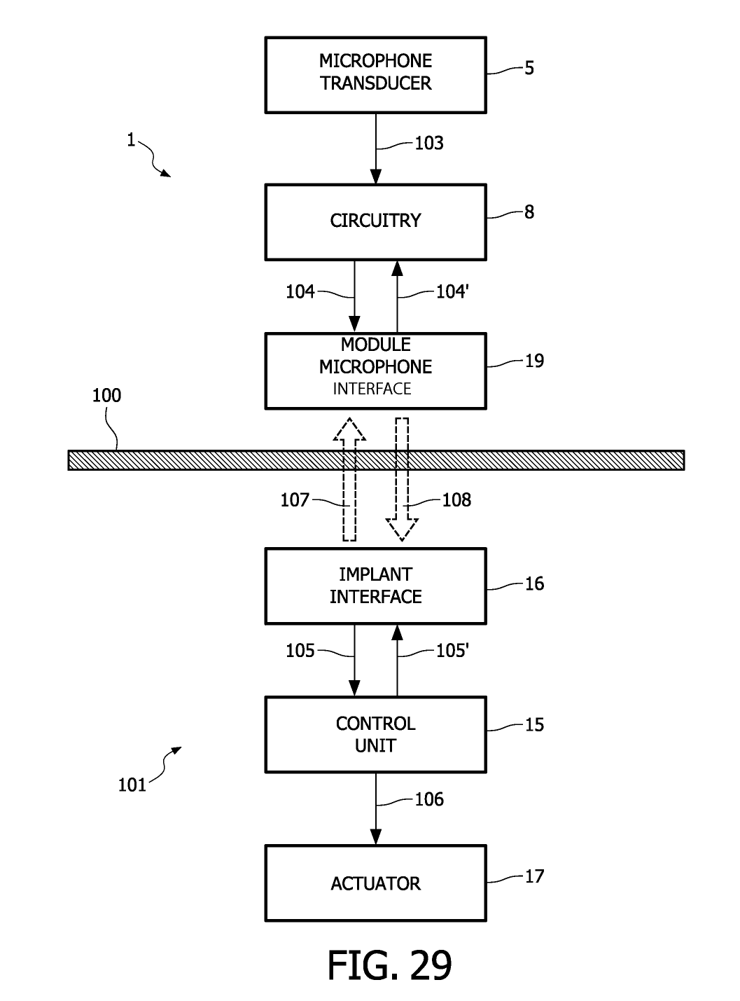

With reference to FIGS. 1 to 6, and 20 to 28, a microphone module 1 of the present invention receives acoustic energy or sound waves collected by the pinna 10 and conveyed via the outer ear canal 12 (FIG. 1) when the module is positioned in the outer ear canal (FIG. 1), or received by the pinna 10 as such (FIG. 21) when the module is positioned thereon. A microphone transducer 5 converts the acoustic energy or sound waves into electrical signals representative thereof; circuitry 8 converts the electrical signals into data signals responsive to the electrical signals, which data signals are received by the implant 15, 16, 17 (101, FIG. 29) as sound information, which implant 101 comprises a control unit 15, an implant interface 16, and actuator 17 which may be an electrode and/or vibration generator. The presence of a wireless interface 19 integrated into the module 1 enables sound information and other data to be transferred to and from the implant without hard wiring; it also enables the module to receive power from the implant. The module, avoiding conventional wiring, may, therefore, be inserted and removed with ease, with or without the need for a specialist.

The location of the microphone module 1 may be in the outer ear canal 12 allowing normal, natural hearing to be mimicked as closely as is technically possible. Moreover, the microphone signal-to-noise performance benefits from the amplifying effect of the hollow resonance that naturally occurs in the external ear canal and boosts the SPL by up to 15 dB around 3 kHz, typically peaking between 1 and 4 kHz, the most crucial frequency band for speech understanding.

The location is not necessarily restricted to the outer ear canal 12, however. As already mentioned, it may attached to any suitable location on the body, especially to the auricle or pinna 10, in particular, the helix 55, scapha 56, concha 57 or lobule 58 (FIG. 21). Sunil Puria (Puria S., “Measurements of human middle ear forward and reverse acoustics: implications for otoacoustic emissions,” J. Acoust. Soc. Am., vol. 113, no. 5, pp. 2773-2789, May 2003) found that in the ear-canal SPL at the tympanic membrane with a vibration generator vibrating the inner-ear fluid in the scala vestibuli, is only 30 dB below the inner-ear SPL. This data suggests that with typical hearing-aid gains of over 40 dB, the sound leaking out of the ear with a middle-ear implant that mechanically vibrates the inner-ear fluid or ossicles, may be loud enough to cause Larsen feedback with a microphone module placed in the outer ear canal 12. In order to reduce the risk of Larsen feedback with a middle-ear implant, the microphone module 1 may placed on the outer ear (auricle or pinna 10) to distance it from the tympanic membrane 11 and reduce the acoustic coupling to the middle ear (FIG. 20). A small opening in the helix 55, scapha 56, concha 57 or lobule 58 (FIG. 21), can be made to securely hold the microphone module. The opening may pass all the way through the helix 55, scapha 56, concha 57 or lobule 58 so forming a passage, or may not pass all the way through, so forming a cavity. The module inductive coil may be implemented on a flexible substrate 59 and be concealed behind the outer ear (FIG. 22). The implant interface 16 may be implanted in the pinna or the vicinity of the pinna 10 rather than the outer ear canal 12 in this case.

Alternatively, the module may be worn flat against the head, for instance held in place by a small magnet in the center of the implant interface. The microphone module is then best implemented as a small flat disk that can be hidden underneath the hair.

With reference to FIG. 29 one embodiment of the invention is a microphone module 1 for use with a hearing implant 101, which module comprises:
a microphone transducer 5,
a wireless interface 19, configured to transfer data signals to and/or from the implant 101, and receive electrical power from the implant 101,
circuitry 8 operably connected to the microphone transducer 5 and interface 19 configured to:
   receive electrical power from the interface 19,
   convert electrical signals generated by the microphone transducer 5 into data signals responsive to the electrical signals, and
   provide data signals to the interface 19, and
a housing 2 that forms a protective body of the module 1, which housing 2 is configured for external attachment to the body. Preferably the housing 2 is configured for concealed insertion in the external ear canal 12, or for attachment to the pinna 10.

### Housing

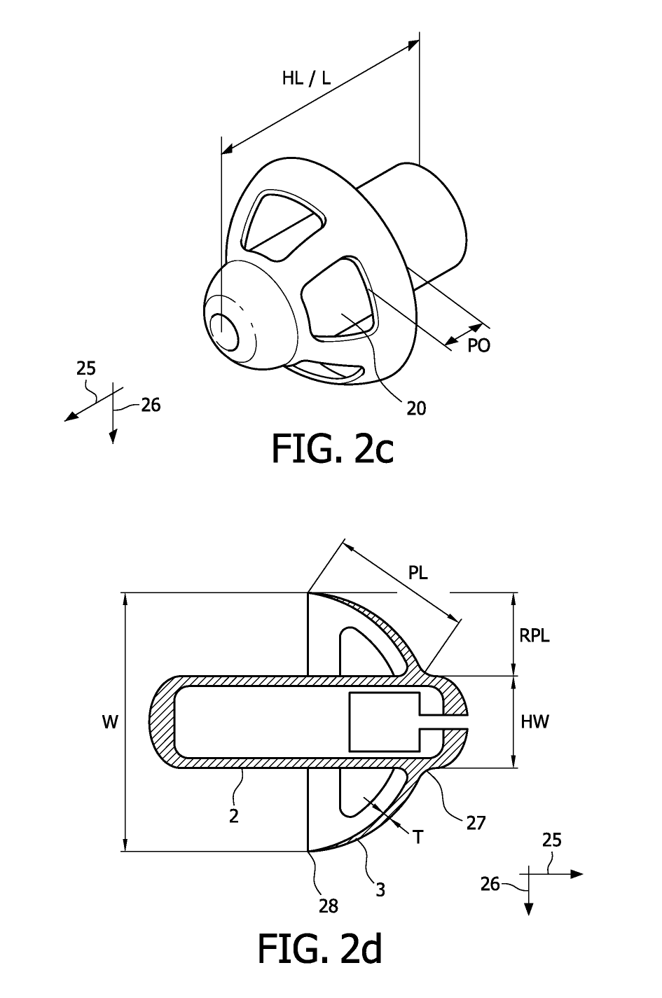

The housing 2 (FIGS. 2a, 2b, 2d to 6, 22 to 28) that encloses the microphone transducer 5, circuitry 8 and other components, provides protection against moisture and prevents foreign material such as cerumen and dust particles from entering the microphone module 1. The size and shape of the housing 2 is generally adapted according to the location where the module 1 is worn. According to one aspect of the invention, the housing 2 may be provided with at least one sound port 4 i.e. a hole through which acoustic energy can reach the microphone transducer 5. The hole may pass all the way through the wall of the housing as shown in the figures, or may pass partially therethrough (not shown). According to one aspect of the invention, the housing 2 is devoid of a sound port 4, being constructed at least partly from an acoustic-transparent material, suitably thin-walled (not shown). Advantageously, there is no requirement for a protective membrane when the housing is formed from an acoustic-transparent material.

The housing is configured for external attachment to the body meaning, it is appropriately dimensioned and shaped according to the area of the body to which it attached. The minimum dimensions of the housing, given elsewhere herein, allow the module to be worn discretely, for example, inserted into the outer ear canal, attached to the pinna or earlobe, which are described in detail below. It is also within the scope of the invention that housing is configured, for example as jewelry stud, for wearing through the nose, lip, eyebrow etc. It may, alternatively, be configured as a press stud for insertion in a reciprocating body cavity introduced for example, on the forehead, chin, cheek or other location.

### Wearing on Outer Ear Canal

Where the module 1 is worn in the outer ear canal 12, an impression mold (not shown) may be made, so that the housing 2 can be adapted to be closely received by the outer ear canal 12 without discomfort to the wearer. If the housing 2 is small enough, flexible buffering structures may allow the size and shape of the housing 2 to be standarised to fit a majority of wearers, and thereby obviating the need for taking an impression mold. Advantageously, the housing is a longitudinal body, having a longitudinal axis 25 and transverse axis 26, preferably adopting an outer capsule, cylindrical or oval shape. It is noted that the longitudinal axis 25 of the module may correspond to a central axis of module 1 (e.g. cylindrical or disk axis) which is also the general direction along which the module 1 is inserted into or withdrawn from the body. The housing 2 should be smaller than or conform to the shape of the outer ear canal 12 to allow a secure fitting and for ease of insertion into and removal from the outer ear canal. To accommodate the natural curvature of the outer ear canal 12, the housing 2 may be curved along its longitudinal axis 25. For individuals with narrow outer ear canals 12, canalplasties could be performed to allow accommodation of the housing 2. In the present invention, the housing 2 may be smaller than the ear canal in transverse 26 cross-section, and the circumferential surface of the housing 2 may be radially extended by one or more buffering structures 3, 3′ that bridge a gap between the inner wall of the outer ear canal and the outer surface of the housing 2. The buffering structure 3, 3′, provides a secure placement of the module 1, and plays a suspension role (see below).

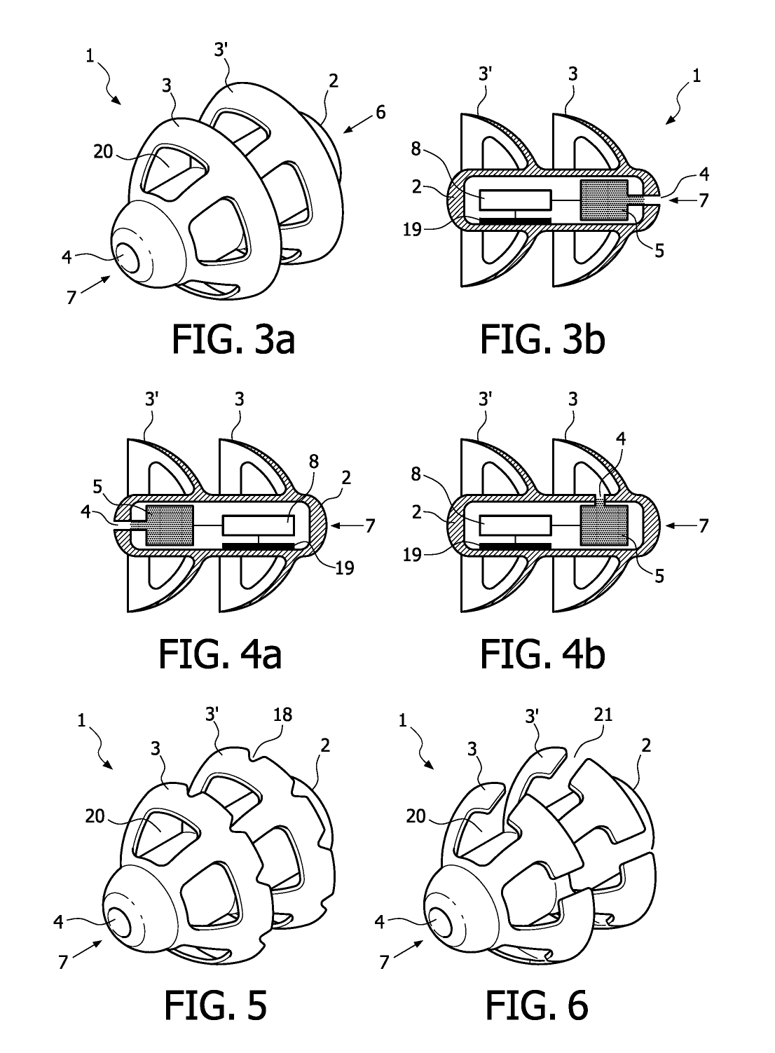

Preferably, the sound port 4 is located at one end of the housing 2 e.g. at the end 7 of the housing 2 facing the pinna 10 as shown in FIG. 4, or at the end 6 of the housing 2 facing the tympanic membrane 11 as shown in FIGS. 2a, 2b, 2c, 2d, 3a and 3b. A sound port 4 located in the longitudinal body of the housing is also within the scope of the invention as shown in FIG. 4b. Preferably, the sound port 4 is located at the tympanic membrane end 7 to maximally benefit from the resonance characteristic of the outer ear canal. For a flatter audio characteristic, the sound port may be located at the pinna end 6 or in the longitudinal body of the housing 2.

When the module is adapted for insertion into the outer ear canal 12, the housing 2, excluding buffering structures 3, 3′ (see below) has a maximum length (HL) and width (HW), the length being measured along a longitudinal or central axis 25 of the housing and the width being measured along an axis 26 perpendicular (transverse) to the longitudinal axis (See FIGS. 2c and 2d). According to one embodiment of the invention, the housing length (HL) is 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm or a value in the range between any two of the aforementioned values, preferably between 5 mm and 15 mm. According to another embodiment of the invention, the housing width (HW) is 0.5 mm, 2 mm, 3 mm, 4 mm, 5 mm, 6 mm, 7 mm, 8 mm, 9 mm, 10 mm, or a value in the range between any two of the aforementioned values, preferably between 3 mm and 5 mm.

### Wearing on Pinna

Where the module 1 is worn on the auricle or pinna 10, the housing 2 will be adapted to attach thereto or to fit into an opening introduced therein. The pinna includes the structure of the helix 55, scapha 56, concha 57 or lobule 58 as shown in FIG. 21. In a preferred embodiment, the housing 2 comprises a longitudinal body, having a longitudinal axis 25 and transverse axis 26, preferably adopting an outer capsule, cylindrical, disc or oval shape. It is noted that the longitudinal axis 25 of the module may correspond to a central axis of module 1 (e.g. cylindrical or disk axis) which is also the general direction along which the module 1 is inserted into or withdrawn from the body. One or both ends of the housing 2 may be disposed with an annular ridge which can act to secure the module 1 to the pinna and/or to contain the wireless module interface 19.

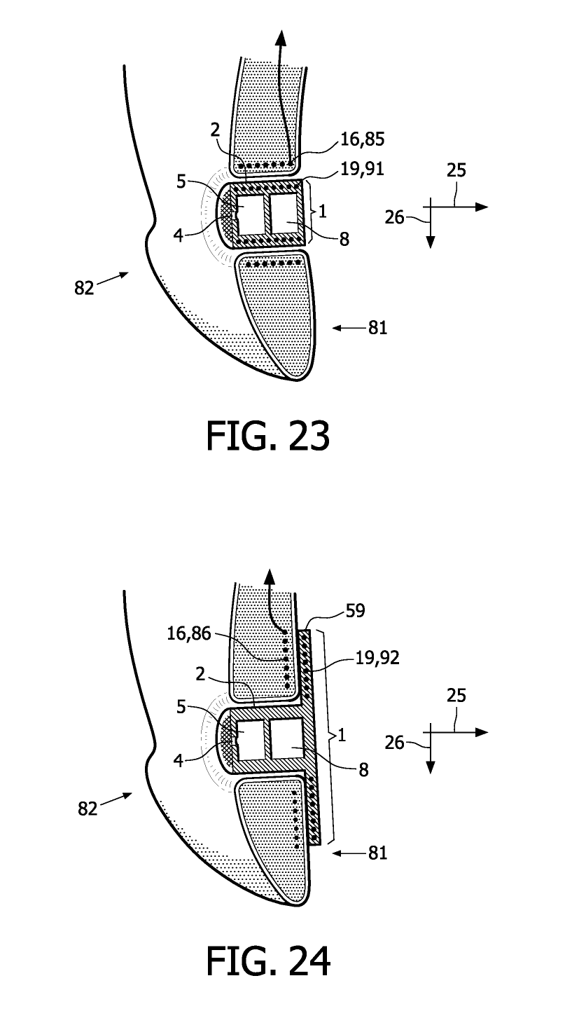

Examples of housing shapes suitable for wearing on the auricle or pinna 10 are depicted in FIGS. 22 to 28. According to one aspect of the invention the dimensions of the cylindrical part of the housing 2 are configured for insertion into an opening in the pinna 10, which opening extends as a passage from the dorsal (back) 81 surface all the way through to the ventral (front) 82 surface of the pinna 10 as shown in FIG. 23. Thus both ends of the cylindrical housing are exposed, the module 1 being worn like a body stud that passes through the pinna 10. According to one aspect of the invention, the housing 2 is devoid of annular ridges. A sound port 4 may be located in a cylindrical end of the housing 2, in the end exposed to the ventral 82 surface (depicted in FIG. 23) or the end exposed to the dorsal 81 surface (not shown).

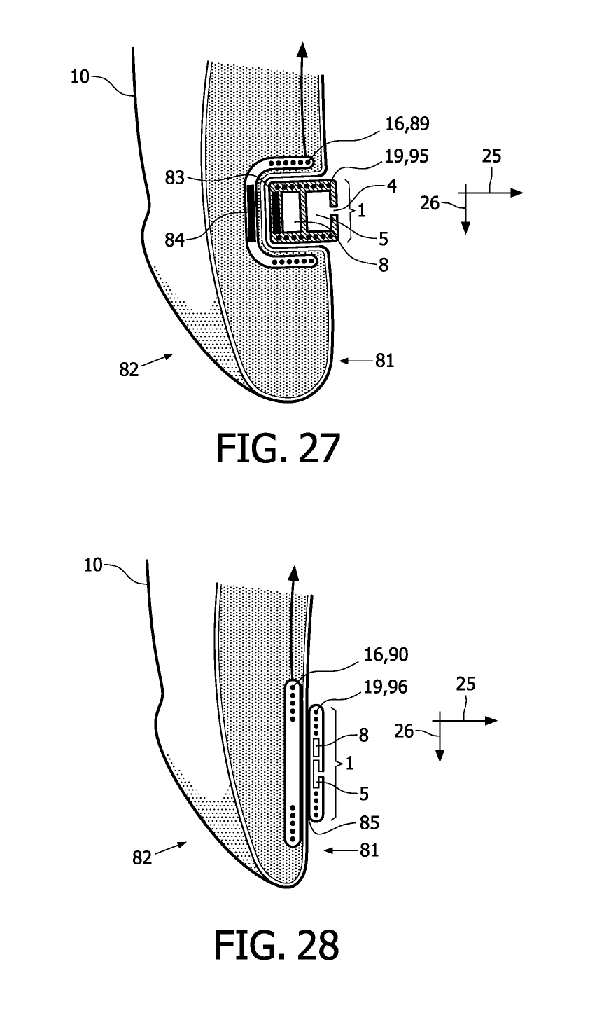

According to another aspect of the invention the dimensions of the cylindrical part of the housing 2 permit insertion into an opening in the pinna 10, which opening is present in the dorsal 81 surface but does not extend all the way through to the ventral 82 surface of the pinna 10 so forming a cavity, as shown in FIG. 27. According to one aspect of the invention, the housing 2 is devoid of annular ridges. A sound port 4 may be located in a cylindrical end of the housing 2, in the end exposed to the dorsal 81 surface (as depicted in FIG. 27). Because the module 1 is only visible from a dorsal 81 (back) view of the subject, said module 1 is discretely worn. A magnet 83 may be present in the cylindrical (ventral 82) end of the module which couples to a magnet 84 implanted beneath the skin of the pinna 10. The function of the magnets is to secure the module 1 during wearing.

According to another aspect of the invention, the module 1 of the invention comprises an essentially cylindrical housing 2, disposed with an annular ridge at one end of the cylinder, which ridge radially extends from the housing body 2 to form a disc-like cap 59 as depicted in FIGS. 22, 24 and 26. The dimensions of the cylindrical part of the housing 2 permit insertion into an opening 80 made in the dorsal 81 surface of the pinna 10. The opening may extend from the dorsal 81 surface all the way through to the ventral 82 surface of the pinna 10 as shown in FIG. 24, so forming a passage. Alternatively, it may not extend all the way through to the ventral 82 surface of the pinna 10 as shown in FIG. 26, so forming a cavity. The disc-like cap 59 may stand proud of and cover the opening 80 in the dorsal 81 surface in situ. The disc-like cap 59 may house the wireless module interface 19 as shown in FIGS. 24 and 26, so providing an enlarged surface area for wireless interactions with implant interface 16, present beneath the skin covering the pinna 10. A sound port 4 may be located in a cylindrical end of the housing 2, in the end disposed with the disc-like cap 59 as depicted in FIG. 26, or in the end devoid of the disc-like cap 59 as depicted in FIG. 24. Because the disc-like cap 59 is only visible from a dorsal 81 (back) view of the wearer, said module 1 is discretely worn.

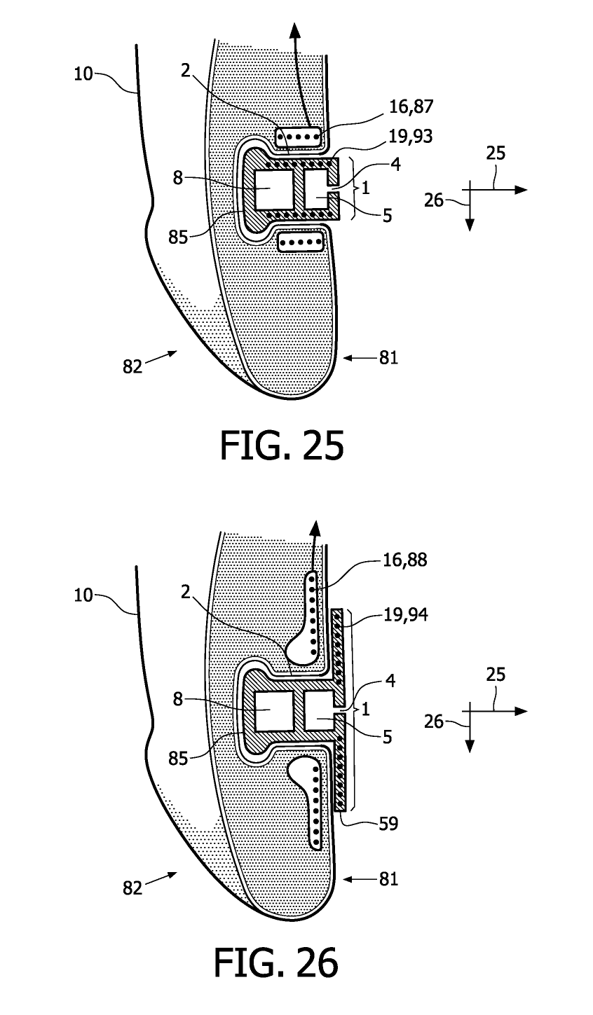

According to another aspect of the invention, the module 1 of the invention comprises an essentially cylindrical housing 2, disposed with an annular ridge at one end of the cylinder, which ridge radially extends from the housing body 2 to form a knob-like protrusion 85 as depicted in FIGS. 25 and 26. The dimensions of the cylindrical part of the housing 2 permit insertion into an opening made in the dorsal 81 surface of the pinna 10. The opening may not extend from the dorsal 81 surface all the way through to the ventral 82 surface of the pinna 10. When the opening is of sufficient size to just accommodate the cylindrical part of the housing 2, the knob-like protrusion 85 will act as a mechanical stop, prevented from passing through the restricted opening, thereby securing the module 1 in the pinna 10. A sound port 4 may be located in a cylindrical end of the housing 2, in the end devoid of the knob-like protrusion 85 as depicted in FIGS. 25 and 26.

When the module is adapted for insertion into an opening in the pinna, the housing 2, has a maximum length (HL) and width (HW), the length being measured along a longitudinal or central axis 25 of the housing and the width being measured along an axis 26 perpendicular (transverse) to the longitudinal axis (See FIGS. 2c and 2d which exemplarily illustrate HL and HW). According to one embodiment of the invention, the housing length (HL) is 1 mm, 2 mm, 3 mm, 4 mm, 5 mm, 6 mm, 7 mm, 8 mm, 9 mm, 10 mm, 11 mm, 12 mm or a value in the range between any two of the aforementioned values, preferably between 2 mm and 12 mm. According to another embodiment of the invention, the housing width (HW), excluding any annular ridges, is 0.5 mm, 2 mm, 3 mm, 4 mm, 5 mm, 6 mm, 7 mm, 8 mm, 9 mm, 10 mm, or a value in the range between any two of the aforementioned values, preferably between 3 mm and 5 mm.

According to another embodiment of the invention, the housing width (HW) in the region of an annular ridge that is a disc-like cap 59, is equal to or more than 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 350%, 400%, 450% or 500%, larger than the HW in the non-ridged region, or a value in the range between any two of the aforementioned values, preferably between 50% and 250% larger. According to another embodiment of the invention, the housing width in the region of an annular ridge that is a disc-like cap 59, is 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm or a value in the range between any two of the aforementioned values, preferably between 5 mm and 15 mm.

According to another embodiment of the invention, the housing width (HW) in the region of an annular ridge that is a knob-like protrusion 85, is 20%, 30%, 40%, 50%, 60%, 70%, 80%, 90%, 100%, larger than the HW in the non-ridged region, or a value in the range between any two of the aforementioned values, preferably between 20% and 70% larger. According to another embodiment of the invention, the housing width (HW) in the region of an annular ridge that is a knob-like protrusion 85, is 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm or a value in the range between any two of the aforementioned values, preferably between 5 mm and 15 mm.

According to one embodiment of the invention, the module 1 of the invention comprises an essentially disc-like housing 2, as depicted in FIG. 28. The disc-like housing 2 provides a surface for contact of the module 1 to the dorsal 81 surface of the pinna 10. The microphone module may be held in place by an adhesive layer 85 or by flat magnets (not shown). The adhesive may be hypo-allergic, suitable for long-term application to human skin. The disc-like housing 2 may stand proud of the dorsal 81 surface. The disc-like housing 2 contains all the components of the module 1, including the wireless module interface 19 as shown in FIG. 28, so providing an enlarged surface area for wireless interactions with implant interface 16, present beneath the skin covering the pinna 10. A sound port 4 may be located in one surface of the housing 2, which surface is not intended for adhesion to the pinna 10. Because the disc-like housing 2 is only visible from a dorsal 81 (back) view of the wearer, said module 1 is discretely worn. RFID tag assembly techniques may be applied to produce this microphone module at very low cost making it a disposable item that can be replaced on a regular basis.

When the module 1 has disc-like housing 2, the housing 2, has a maximum length (HL) and width (HW), the length being measured along central axis 25 of the disc and the width being measured along an axis 26 perpendicular (transverse) to the longitudinal axis (See FIGS. 2c and 2d which exemplarily illustrate HL and HW). According to one embodiment of the invention, the housing length (HL) is 1 mm, 2 mm, 3 mm, or 4 mm or a value in the range between any two of the aforementioned values, preferably between 1 mm and 3 mm. According to another embodiment of the invention, the housing width (HW), excluding any annular ridges, is 3 mm, 4 mm, 5 mm, 6 mm, 7 mm, 8 mm, 9 mm, 10 mm, 11 mm, 12 mm, 13 mm, 14 mm, 15 mm or a value in the range between any two of the aforementioned values, preferably between 5 mm and 13 mm.

The body of the housing 2 is made from or coated with a biocompatible material for example, surgical steel, platinum, iridium, titanium, gold, silver, nickel, cobalt, tantalum, molybdenum, or their biocompatible alloys. It may, alternatively, be made from or coated with a biocompatible polymer such as PTFE or silicone polymer. It may alternatively or in addition be coated with a hydrophobic layer; such layer acts to repel water from the exposed surfaces and thus reduce the possibility of water migrating into the sensitive microphone transducer and electronics. According to one aspect of the invention, the housing is hermetically sealed, apart from a sound port 4 hole where present.

### Protective Membrane

A thin, audio transparent, permanent or replaceable protective membrane may be disposed over the sound port 4. This acts to protect the sound port 4 and microphone transducer 5 by preventing foreign material such as cerumen, from entering the microphone module 1. Thus, one embodiment of the invention is a microphone module provided with such a replaceable membrane over the sound port. In one aspect of the invention, the membrane is made out of a metalized polymer, or metal. Metalized polymers may be made from film or sheets of polymer including poly (arylene ether), polyamide, polyimide, polyester, and polyolefin thin film, as well as their copolymer, blend, and composite thin films combined with metals such as aluminum, or zinc in process known in the art. Suitable metals for a protective membrane include stainless steel, titanium, silver, platinum, or gold. According to another aspect of the invention, the protective membrane is coated with a hydrophobic layer. In yet another aspect in the invention, the protective membrane is preferably made from a material having the requisite audio conducting properties, as well as being gas permeable and waterproof so preventing solids and liquids from entering the module, but allowing gaseous exchange for water vapour release and barometric relief. According to a preferred aspect of the invention, the protective membrane is made from Gore-Tex® (marketed by W. L. Gore & Associates). Other suitable materials include Gore-Tex XCR with improved breathability, eVent breathable fabrics manufactured and marketed by the BHA Group, Inc., and Entrant breathable fabrics by Toray Industries, Inc., Japan.

The skilled person will understand that the membrane will be sufficiently thin to vibrate in concert with sound entering the outer ear canal. The precise thickness will depend on the material used and the size of the sound port hole, however as a guide, a protective membrane will have a thickness of between 1 μm, 2 μm, 5 μm, 6 μm, 8 μm, 10 μm, 15 μm, 20 μm, 25 μm, 30 μm, 40 μm, 50 μm, 60 μm, 70 μm, 80 μm, 100 μm, 200 μm, 300 μm, 400 μm, 500 μm, 1 mm or a value in the range between any two of the aforementioned values, preferably between 1 and 100 μm.

The protective membrane is not just limited to the microphone module of the present invention; it may incorporated in any body worn microphone of the art allowing passage of sound to a microphone transducer while preventing foreign material such as cerumen, from entering the microphone.

Another embodiment of the invention is a placement tool configured to allow attachment and/or removal the replacement protective membrane from the module 1.

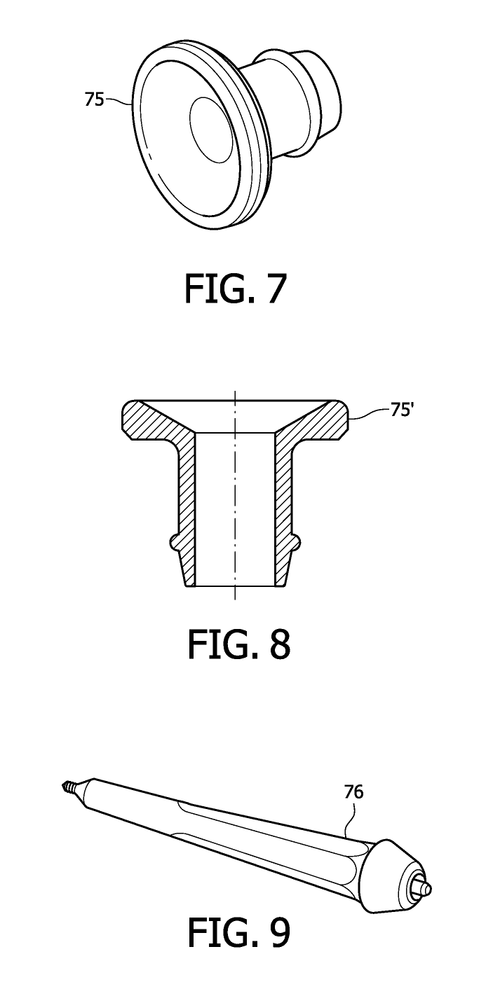

According to another aspect of the invention, the protective membrane is a C-barrier™ marketed by Sonion, which is a funnel-shaped membrane having suitable characteristics for use with the present module. C-barrier™ is an example of a user-replaceable membrane 75 (FIG. 7), 75′ (FIG. 8). The C-barrier™ membrane may be mounted on a placement tool 76 (FIG. 9) that protects the fragile membrane during transport and application. The user first removes the old membrane using the one end of the placement tool 76 and then installs the new membrane using the other tool 76 end. The user then releases the tool leaving the membrane 75, 75′ properly seated and protected in the sound port.

The invention includes use of a placement tool 76 for inserting and removing a C-barrier™ from a microphone module 1 of the invention. Particularly, it includes the use of such placement tool as manufactured and marketed by Sonion for inserting and removing a C-barrier™ from a microphone module 1 of the invention.

### Buffering Structures

As mentioned above, when the module is for insertion into the outer ear canal 12, at least part of the cylindrical body of the housing 2 may be radially extended with one or more buffering structures 3, 3′ as depicted in FIGS. 1 to 6 and 10, 11 and 15. The buffering structures 3, 3′ are configured to bridge a gap between the inner wall of the outer ear canal 12 and the outer surface of the housing 2 body in situ. Description of the housing 2 herein may thus include the buffering structures 3, 3′.

A buffering structure 3, 3′, is made of a flexible material, which can bend and/or compress upon insertion into the outer ear canal 12. The profile of the module 2, viewed along the longitudinal axis 25, and extended by the buffering structure 3, 3′, is slightly larger than the width of the outer ear canal 12. Thus, the buffering structure 3, 3′ provides a secure placement of the module 1 by providing one or more points of friction against the wall of the outer ear canal.

The buffering structures 3, 3′ also function to centre the sound port 4 in the outer ear canal 12 so that the sound port does not make contact with the wall of the outer ear canal 12. The result is that vibrations emanating from the outer ear canal 12 are less likely to be received by the microphone transducer 5. In addition to providing a securing and centering role, the buffering structures 3, 3′ also act to cushion the housing 2 from shock movements.

In a preferred embodiment of the invention, a buffering structure 3, 3′ is an annular ring extending from the circumferential surface of the housing 2. The annular structure is of sufficient width to bridge a gap between the housing and the wall of the outer ear canal 12. Preferably the buffering structure 3, 3′ an annular ring, the thickness of the ring allowing sufficient flexibility of the buffering structure that the module can be comfortable worn without an undue feeling of pressure.

The thickness, T (see FIG. 2d), of the buffering structure 3 is the minimum distance measured in a straight line from a point on one surface of the buffering structure, through the buffering structure 3, to the other surface; T is preferably measured along a cross-section of the module 1 though the longitudinal axis 25 as depicted in FIG. 2d. Advantageously, T may be 0.1 mm, 0.2 mm, 0.4 mm, 0.6 mm, 0.8 mm, 1 mm, 2 mm, 3 mm, 4 mm, or 5 mm or a value in the range between any two of the aforementioned values, preferably between 0.2 and 1 mm. The value of T may vary, being smaller towards the extremity 28 of the structure 3 compared with at the base 27. The base 27 is the region of attachment of the buffering structure 3 to the body of the housing 2, and the extremity 28 is the outermost point of the buffering structure 3.

The protrusion length, PL (see FIG. 2d), of the buffering structure 3 is a distance measured in a straight line from the base 27 to the extremity 28 of the buffering structure; PL is preferably measured along a cross-section of the module 1 though the longitudinal axis 25 as depicted in FIG. 2d. PL may be no greater than 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm, or a value in the range between any two of the aforementioned values, preferably between 4 and 10 mm.

The radial protrusion length, RPL (see FIG. 2d), of the buffering structure 3 is a radial distance measured from the base 27 to the extremity 28 of the buffering structure; the RPL is preferably measured along a cross-section of the module 1 though the longitudinal axis 25 as depicted in FIG. 2d. The RPL may be no greater than 1.25 mm, 1.5 mm, 2 mm, 3 mm, 4 mm, 5 mm, 6 mm, 7 mm, 8 mm, 9 mm, 9.75 mm or a value in the range between any two of the aforementioned values, preferably between 1 and 8.5 mm.

In a preferred embodiment, the buffering structure 3, 3′ is an annular ring angled to the longitudinal body of the housing 2 so as to give a conical appearance, whereby the apex part of the cone points towards the tympanic membrane end 7 of the housing 2. The angled annular buffering structure is generally attached at its base 27 towards the tympanic membrane end 7 of the housing 2, and it circumferentially extends outwards and backwards towards the direction of the pinna end 6 of the housing 2. This creates a buffering structure having the appearance of a conical flap as shown in the figures. The extremity 28 of the angled annular buffering structure can thus be compressed radially with the application of light pressure. The skilled person will understand that an angled annular ring is optimised for insertability and security. Since the outer ear canal often exhibits an oval instead of circular cross-section, buffering structures with an extremity 28 having an oval shape when viewed along the longitudinal axis may prevent or reduce module rotation in situ.

The number of buffering structures 3, 3′ may be 1, 2, 3, 4, 5, 6, 7 or more, preferably aligned along the longitudinal axis 25 of the housing body. An embodiment having one buffering structure 3 is shown in FIGS. 2a and 2b; embodiments having two buffering structures 3, 3′ are shown in FIGS. 3a, 3b, 4a, 4b, 5 and 6. Generally, the greater the number of buffering structures, the better the alignment and security, however, this needs to be balanced with increased weight, bulk and cost of production. The preferred number of buffering structures 3, 3′ is two.

According to one aspect of the invention, the base 27 of a buffering structure 3, 3′ is disposed over a central part of the housing body. The central part of the housing body may be defined as the region that extends both in the direction of tympanic membrane end 7 and pinna end 6 of the housing 2, from an imaginary circumferential line equidistant from said ends, by an amount that is 30%, 33%, 40%, 50%, 60%, 67%, 70%, 80%, 90 or 95%, preferably by 10 to 95% of the longitudinal length, HL, of the housing.

Preferably a buffering structure 3, 3′ is provided with one or more perforations 20 to allow the passage of sound waves therethrough to reach the tympanic membrane 11. This reduces the booming-voice effect that some wearers experience when their outer ear canal 12 is sealed. The perforations 20 also serve to ventilate the space between the microphone module 1 and the tympanic membrane 11 that would otherwise be sealed off increasing the risk of skin reactions and infection.

The perforation offset, PO (see FIG. 2c), of the buffering structure 3 is a distance measured along the longitudinal axis 25 from the extremity 28 of a buffering structure 3, 3′ to the edge of a perforation 20. The PO may be 1.0 mm, 1.25 mm, 1.5 mm, 2 mm, 3 mm, 4 mm, 5 mm, 6 mm, 7 mm, 8 mm, 9 mm, 9.75 mm, 10 mm or a value in the range between any two of the aforementioned values, preferably between 2 and 5 mm.

The perforation 20 may extend 21 to the extremity of the buffering structure 3, 3′ as shown in FIG. 6, so allowing the radial width of the buffering structures (and the width of the module 1) to decrease more readily under circumferential compression, and permitting the passage of cerumen past the module in situ. In such case, the PO is zero.

In addition or besides perforations 20, the extremity of a buffering structure may be disposed with one or more notches 18 as shown in FIG. 5. These also allow the radial width of the buffering structures (and the width of the module 1) to decrease more readily under circumferential compression, and permit the passage of cerumen past the module in situ.

The buffering structures 3, 3′ are typically made from a flexible material such as medical-grade silicone, medical grade rubber, or other suitable polymer. According to one aspect of the invention, materials having durometers of about 20 to 60 Shore A, preferably about 25 Shore A provide the desired flexibility.

According to one aspect of the invention, the module 1, including buffering structures 3, 3′ has a length (L) and width (W), the length being measured along a longitudinal axis 25 of the housing and the width being measured along an axis 26 perpendicular (transverse) to the longitudinal axis (See FIGS. 2c and 2d). According to one embodiment of the invention, the length (L) of the module 1 is 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm or a value in the range between any two of the aforementioned values, preferably between 5 mm and 15 mm. According to another embodiment of the invention, the width (W) of the module 1 is 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm or a value in the range between any two of the aforementioned values, preferably between 5 mm and 15 mm.

### Microphone Transducer

The microphone transducer 5 of the invention is positioned in the module 1 so that its sound receiving end is directed towards a sound port 4 or towards an acoustic transparent region of the housing 2 wall.

Where the module 1 is for insertion into the outer ear canal 12, the microphone transducer 5 may be positioned in the module so that its sound receiving end is directed towards the pinna end 6 of the module as shown in FIG. 4a. Alternatively, the microphone transducer 5 may be positioned in the module so that its sound receiving end is directed towards the tympanic membrane end 6 of the module as shown, for example, in FIGS. 2a, 2b, 2c, 2d, 3a and 3b. According to still another alternative, the microphone transducer 5 may be placed in the module so that its sound receiving end is directed towards the longitudinal body of the module 1 as shown, for example, in FIG. 4b.

Where the module 1 is for insertion into an opening introduced in the pinna 10, or attachment thereto, the microphone transducer 5 may be positioned in the module so that its sound receiving end is directed towards the dorsal 81 facing end of the module as shown in FIGS. 25 to 28. Alternatively, the microphone transducer 5 may be positioned in the module so that its sound receiving end is directed towards the ventral 82 facing end of the module as shown in FIGS. 23 and 24.

Advantageously, the microphone may be an electret microphone, such as manufactured by the Knowles Corporation. Miniaturized microphones are available in a variety of configurations including omni-directional, matched omni-directional, and unidirectional.

### Module Interface

The respective module 19 interfaces are configured to transfer data signals between module and implant, and to transfer power from the implant to the module by avoiding a hard-wired electrical contact between the implant and module. Configured means the interfaces comprise the necessary components, such as elaborated below (e.g. induction coils, capacitative plates, electrical contacts, light sources and sensors), positioned and set up for optimum performance. A problem in the prior art is that electrical contacts, where wires pass from beneath the skin to the surface are prone to infection, induce irritation and may cause pain when the module is inserted. The module interface 19, on the other hand, utilizes wireless alternatives. The person skilled in the art will understand the variety of means available for wireless transfer of data and/or power between two devices, and how to implement them. By way of a preferred embodiment and elaborated further is an interface 19 of the microphone module 1 configured to receive electrical power using magnetic (inductive) coupling, conductive coupling, non-conductive (i.e. capacitive) coupling and/or optical coupling. These four means for receiving electrical power may also be used to exchange data signals between the implant to the microphone module 1 as will become clear.

### Inductive Coupling

Where an inductive coupling is used to transfer electrical power and/or transfer data signals between the implant and microphone module 1, the module interface 19 comprises one or more induction coils. Said coil is inductively energized by a reciprocating induction coil of the implant. The use of induction to transfer electrical power wirelessly is well known in the art for example, from Schuder J. C., et al, “High-level electromagnetic energy transfer through a closed chest wall,” IRE Int. Cony. Record., vol. 9, pp. 119-126, 1961; Ko W. H., et al. “Design of radio-frequency powered coils for implant instruments,” Med. & Biol. Eng. & Comput., vol. 15, pp. 634-640, 1977; Donaldson N. de N. “Analysis of resonant coupled coils in the radio frequency transcutaneous links,” Med. & Biol. Eng. & Comput., vol. 21, pp. 612-627, 1983.

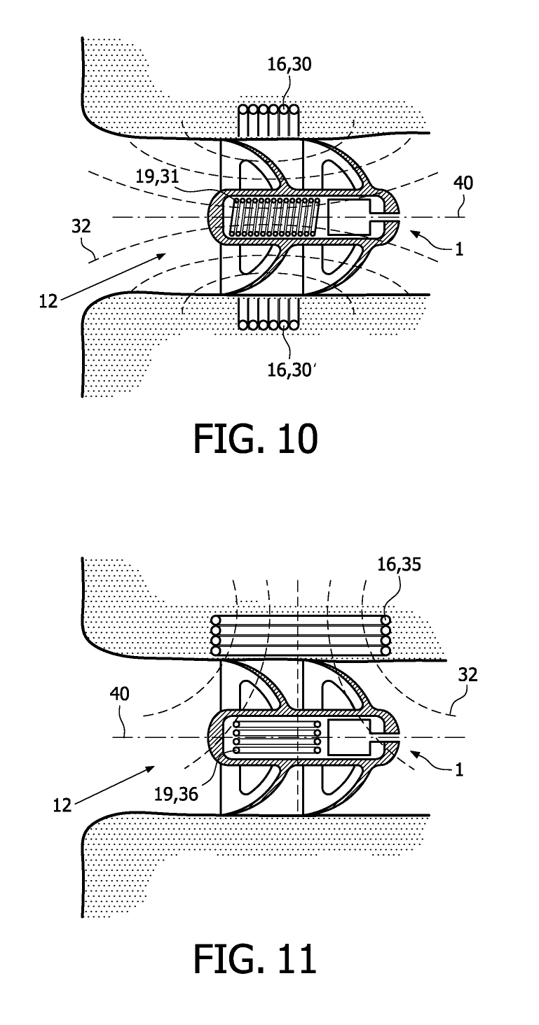

FIGS. 10, 11, 14 and 23 to 28 illustrate an embodiment of the invention, showing an arrangement of module induction coils and implant induction coils. According to this embodiment, the module interface 19 comprises a module induction coil 31, 36, 91, 92, 93, 94, 95, 96 having an axis of winding approximately parallel to the axis of windings of the implant interface 16 induction coil 30, 30′, 35, 85, 86, 87, 88, 89, 90. Within the module, an axis of winding of the module interface 19 induction coil may be essentially parallel to the longitudinal axis 25 of the module 1 (parallel alignment, e.g. FIGS. 10, 23 to 28), or may be essentially perpendicular to the longitudinal axis 25 of the module 1 (longitudinal alignment, e.g. FIGS. 11, 14, 15).

### Inductive Coupling—Parallel Alignment

According to one embodiment of the invention, the axis of winding of the module induction coil 31, 91, 92, 93, 94, 95, 96 is essentially parallel to the longitudinal axis 25 of the module 1 (FIGS. 10, and 23 to 28); the cylindrical shape of the housing naturally accommodates the loops of the coils. In the parallel alignment, the coils may be disposed in a tubular configuration or planar configuration within the microphone module.

### Inductive Coupling—Parallel Alignment—Tubular Coils

The tubular configuration is where the axis of winding of the microphone module coil is essentially parallel to the longitudinal axis 25 of the module 1, and the coil also extends in a longitudinal direction along the housing 2. The result is a coil having a tubular shape. The tubular arrangement permits an essentially coaxial juxtaposing of the module interface 19 coils 31, 91, 93, 95 (FIGS. 10, 23, 25, 27) and the implant interface 16 induction coils 30, 30′, 85, 87, 89 in situ, so providing a natural coil alignment and a strong coupling. It is preferred that the module interface 19 coils are surrounded by the implant interface 16 induction coils in situ in an essentially concentric configuration, but it is not a requirement. For example, the module interface 19 coils may be non-concentric but parallel and coaxial the implant interface 16 induction coils in situ, and still provide exchange of data and power. Generally, the implant interface 16 induction coils 30, 30′, 85, 87, 89 have a tubular shape along at least part of the passage of the outer ear canal 12. The parallel and coaxial alignment is insensitive to module rotations around the longitudinal axis, allowing some tolerance regarding the insertion orientation.

### Inductive Coupling—Parallel Alignment—Planar Coils

The planar configuration is where the axis of winding of the microphone module coil is essentially parallel to the longitudinal axis 25 of the module 1, and the coil extends in an annular direction outwards from the centre of the coil. The result is a coil having a flat, planar shape. The planar arrangement permits a configuration of planar coils whereby implant interface 16 induction coils 86, 88, 90 at least partly overlap the module interface 19 coils 92, 94, 96 (FIGS. 24, 26 and 28) in situ, but where the implant interface coil does not surround the module interface coil. The overlap may be coaxial, essentially coaxial or not coaxial. The parallel configuration of module 1 planar coils is also insensitive to module rotations around the longitudinal axis. The implant interface 16 induction coils 86, 88, 90 may have a planar shape as shown in FIGS. 24, 26 and 28. Alternatively, implant interface 16 induction coils may have a tubular shape (not shown).

### Inductive Coupling—Perpendicular Alignment

FIG. 11 depicts another configuration of the invention, whereby the axis of winding of an induction coil 36 of the module interface 19 is aligned essentially perpendicular to the longitudinal path 40 of the outer ear canal 12. In other words, the axis of winding of the module coil 36 is essentially perpendicular to the longitudinal axis 25 of the module 1. The perpendicular arrangement also permits a coaxial configuration of coils whereby implant interface 16 induction coils 35 at least partly overlap the module interface 19 coils 36 (FIG. 11) in situ. The so called perpendicular configuration eases surgical demands in respect of an outer-ear worn module, requiring the insertion of a single induction coil 35 along the ear canal without having to cut the skin lining the ear canal.

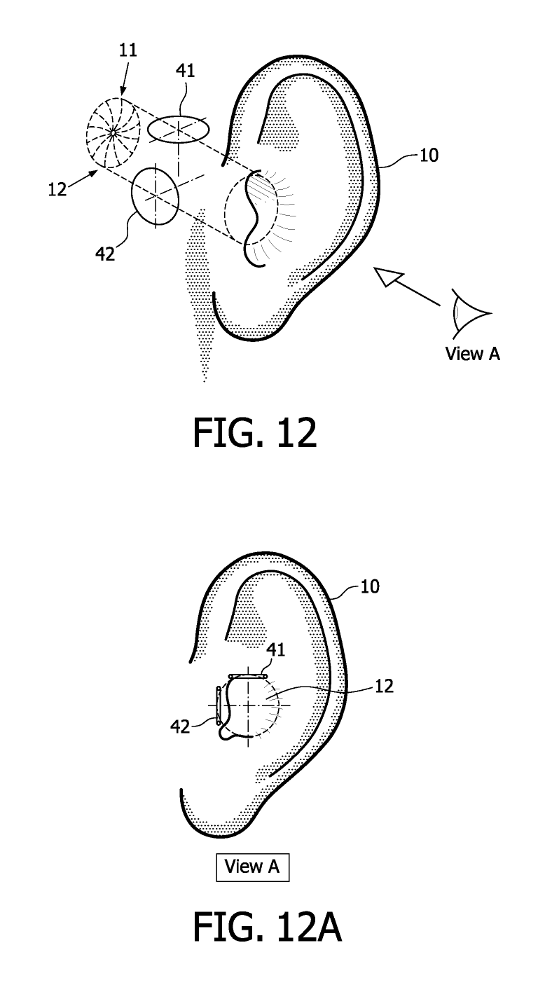

The perpendicular arrangement is, however, sensitive to module rotations around the longitudinal axis respect to the implant interface coil. This, however, can be alleviated by the use of two essentially orthogonal coils in the implant interface 16 in order to generate a rotating field. An orthogonal coil arrangement comprises two induction coils 41, 42 placed in the outer ear canal 12 so that their axis of winding are essentially at 90 deg as shown in FIGS. 12 and 12A. By supplying the orthogonal coils with 90 deg out-of-phase AC currents, a rotating field may be created by the implant interface 16. This arrangement desensitizes the system to axial rotations of the microphone module.

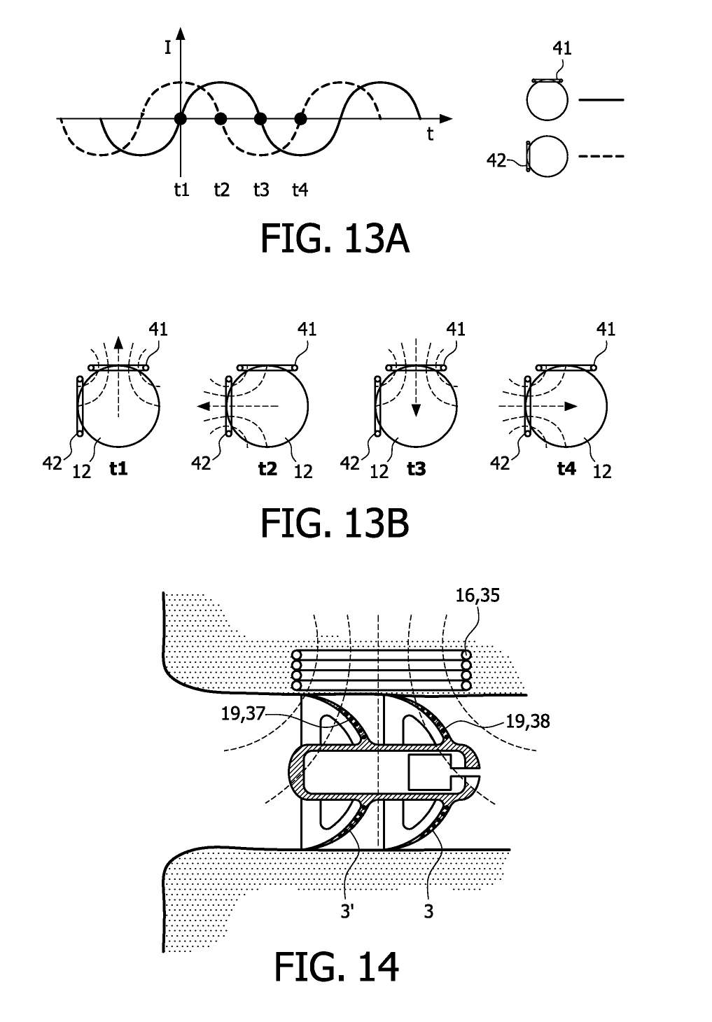

The creation of a rotating field using orthogonal coils is a principle known in the art, and is within the practices of the skilled person. For the sake of completeness, however, a short description follows. The creation of a rotating magnetic field by means of orthogonal coils and 90 deg out-of-phase AC currents is illustrated in FIGS. 13A and 13B. FIG. 13A depicts the phase of the AC current (I) over time (t) passing through each of the orthogonally implanted coils 41 (solid line) and 42 (dashed line), and FIG. 13B shows the net direction and orientation of the magnetic field generated by the coils at time points t1 to t4. At time point t1 the coil 41 carries a maximum positive current (FIG. 13A) so producing a magnetic field pointing upwards in FIG. 13B. The coil 42 carries a zero current at this point. As time progresses, the current passing through coil 41 decreases while the current passing through its orthogonal counterpart 42 increases, up to a point t2 where the coil 41 has zero current passing through and the coil 42 carries a maximal and positive current so producing a net magnetic field pointing left wards (FIG. 13B). With equal coil current amplitudes and orthogonal coils, the net magnetic field does not change magnitude between t1 and t2; it only rotates in space 90 deg counterclockwise. This process continues through the remaining 180° (t3) and 270 (t4) time points, thereby completing the 360° counterclockwise rotation of the magnetic field. The induction coil present in the microphone module will be induced maximally when the rotating magnetic field of the implant interface 16 momentarily aligns with the axis of winding of the microphone module interface coil 19. As can be deduced from FIG. 13B, alignment would occur twice in every complete rotation of the magnetic field, once in a direction inducting a positive coil voltage, and once in the opposite direction inducing a negative coil voltage.

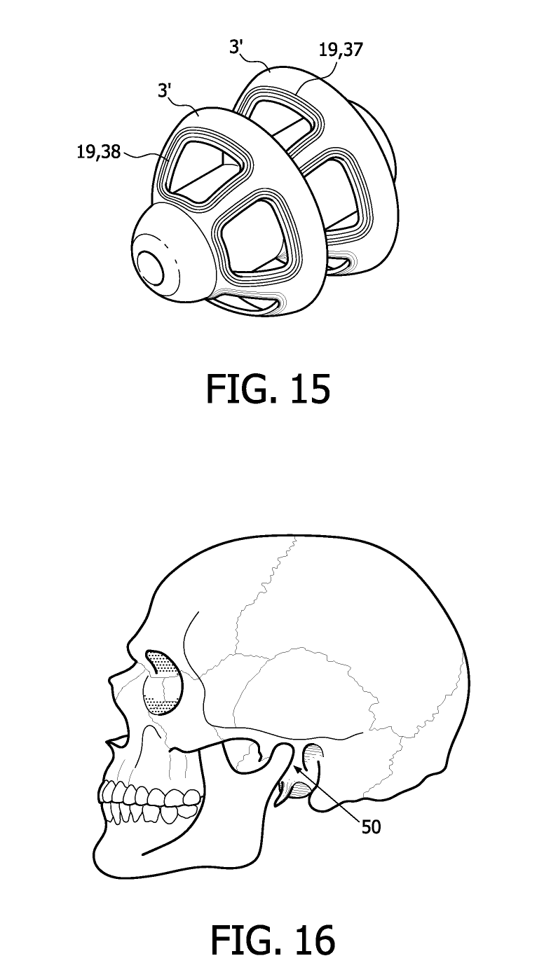

As a variation on the perpendicular coil alignment, the coils 37, 38 of the module interface 19 are comprised in the buffering means 3, 3′ as shown in FIGS. 14 and 15. This brings the implant interface 16 coils 35 and module interface 19 coils 37, 38 closer together, improving the power efficiency of the inductive coupling.

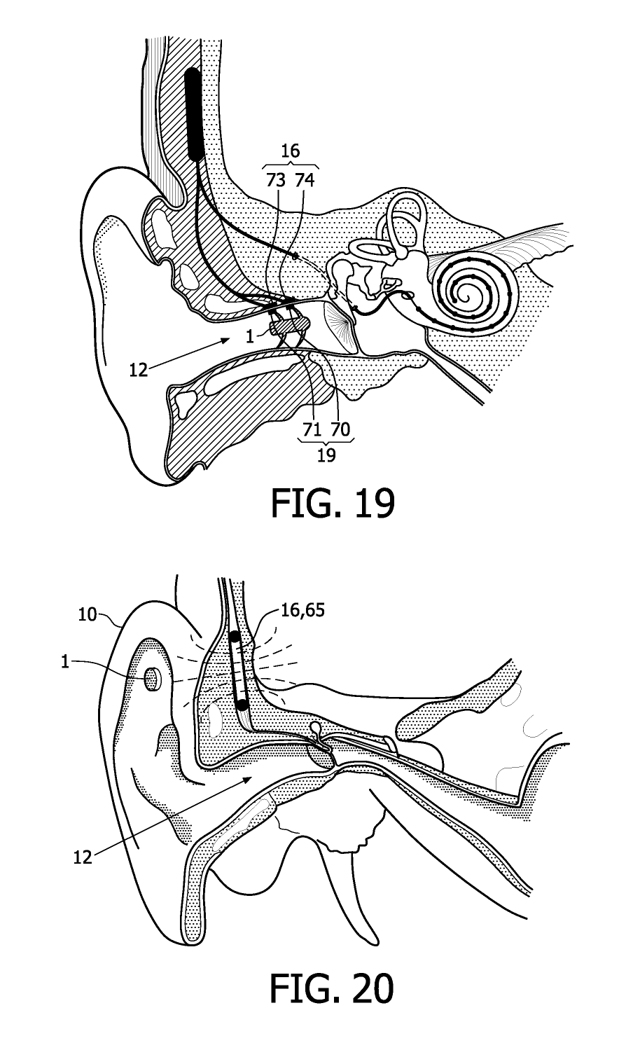

### Conductive Coupling

Where conductive coupling is used to transfer electrical power and data signals between the implant and microphone module 1, the module interface 19 comprises one or more electrical contacts which provide an electrical connection between the implant and the microphone module 2. It is noted that the implant contacts are placed below the skin, meaning they are not in mechanical contact with the microphone module 2. An embodiment having this feature is depicted in FIG. 19 which shows a microphone module 1 of the invention, wherein the module interface 19 is a pair of electrical contacts 70, 71, and the implant interface 16 comprises a reciprocating pair of electrical contacts 73, 74 subcutaneously. The contacts are placed on the body of the housing 2, and are spatially separated. The contacts are positioned so that they will make contact with the wall of the outer ear canal 12. Preferably the contacts are placed on the buffering means 3, 3′, one contact one each buffering means.

### Capacitive Coupling

A capacitive connection may be formed by parallel-plate capacitors with one pair of plates implanted below the skin and another pair of plates in the microphone module. Where such capacitive coupling is used to transfer electrical power and data signals between the implant and microphone module 1, the module interface 19 comprises a pair of capacitive plates, and the implant interface 16 comprises of a reciprocating pair of plates, forming two side-by-side capacitors. Said plates may be arranged so that their reciprocating surfaces substantially overlap each other. A capacitive connection may be formed when capacitance is formed between the module capacitor plates and reciprocating capacitor plates implanted in the vicinity of the module insertion region. An embodiment having this feature is may be understood from FIG. 19 whereby the electrical contacts 70, 71, 73, 74 can each be replaced by capacitive plates, seen here arranged side-by-side.

The plates may be made at least partly from or coated with, for example, surgical steels, or platinum, iridium, titanium, gold, silver, nickel, cobalt, tantalum, molybdenum, or their biocompatible alloys, coated to lower their DC and/or AC impedance; examples of suitable coatings include porous platinum, titanium nitride with or without carbon, iridium, iridium oxide, titanium nitride with iridium oxide, or tantalum-based coatings.

A reciprocating pair of capacitative plates is commonly formed from two flat, planar surfaces, one flat planar surface in the implant interface, the other in the module interface. However, the invention is not limited to these shapes, other configurations being possible such as curved plates. The surfaces may adopt rectangular, square, circular polygonal, or irregular shapes. The plates in the module interface are preferably arranged side by side, however, it is also within the scope of the invention that they are concentrically arranged.

### Optical Coupling

Since tissue and skin are to some extent transparent, especially in the infrared band, light (e.g. infrared, visible) can be used to transfer power and/or data through a skin and tissue layer. Where optical coupling is used to transfer electrical power and/or transfer data signals from the implant 101 to the microphone module 1, the implant interface 16 comprises a light source, and the module interface 19 a photovoltaic cell that converts captured light flux in electrical current.

For the transfer of data in the reverse direction—from the microphone module to the implant—the implant interface 16 further comprises a light sensor and the module interface 19 further comprises a light source. The light source in the module interface 19 will change in the intensity and/or the wavelength of emitted light responsive to the data. The light sensor in the implant interface 16 will detect changes in intensity and/or wavelength of emitted light enabling data to be read by the implant. It will be the understood that the skilled person may readily implement an optical coupling in accordance with the invention using practices standard in the art.

### Transfer of Data Signals

The circuitry 8 of the invention converts the electrical signals produced by the microphone transducer 5 into data signals responsive to the electrical signals. The electrical signals represent audio information; said information is transferred as data signals from the interface 19 present in the module 1 and is received by an implant interface 16 to be converted into electrical signals to stimulate electrodes and/or mechanical transducer implanted in the ear. The data signals transferred by the module 1 may comprise other information besides audio information, such as operating parameters (e.g. gain settings), environmental information (e.g. temperature, moisture, sound level), which information can be used in a feedback loop to adjust settings in the module 1 or to change how the data signals are converted into electrical/mechanical stimulations in the implant 101.

Data signals may also be employed to send information from the implant to the module 1, such as operating parameters (e.g. gain settings), mode settings (e.g. microphone, telecoil, infrared, radio—see below) which information can be used to adjust settings in the module 1.

It will become apparent that the present invention reverses the typical relationship between the ‘master’ and ‘slave’ components of a hearing system. Devices of prior art typically comprise an externally worn ‘master’ hearing-aid processor and an implanted ‘slave’ actuator or electrode that only generates the stimuli for the auditory system. The present invention reverses that relationship, with the ‘master’ hearing-aid processor and actuator both implanted in the patient body, and the ‘slave’ ear-canal unit only providing the audio information.

The data signals may take any suitable form, depending on the required energy consumption, cost restrictions, and the degree of interference tolerated. Examples of suitable data signals include, for example, amplitude modulated signal, frequency modulated signal, phase modulated signal, pulse-width modulated signal, pulse sequence, pulse sequence with an SPL-depending frequency, pulse sequence with an SPL-depending pulse width, pulse sequence with an SPL-depending pulse phase, or a digitally encoded pulse sequence. The audio signal generated by the microphone transducer 5 may be dynamically compressed prior to modulation to improve the signal to noise performance.

Preferably, the wireless interfaces 16, 19 use magnetic (inductive) coupling, conductive coupling, non-conductive (i.e. capacitive) coupling, optical (e.g. infrared) coupling or any combination thereof.

### Inductive Coupling

According to one aspect of the invention, inductive coupling is used to transfer data signals from the implant to the microphone module 1 and/or vice versa. Using an inductive coupling, the implant interface 16 comprises an inductive coil that generates a magnetic flux using AC current, which flux is pickup up wirelessly by the microphone module 1 interface 19 which is a coil in which an AC voltage is induced.

AC voltage induced in the microphone module 1 coil 31, 36, 37, 38, 91-94 is rectified to a DC voltage, which DC voltage may be used to power the microphone circuitry. The use of backscattering may be employed to transfer data from the microphone module 1 to the implant 101 using, for example, load shift keying (LSK) or phase shift keying (PSK).

In LSK, the load current of the microphone circuitry 8 is modulated, which leads to modulation in the magnetic coupling field, and hence the implant inductive coil 30, 35, 41, 42, 60, 61, 62, 63, 65, 85-90 voltage. The microphone module 1 transfers data signals to the implant in this manner, such data being, for example, sound received by the microphone transducer 5. As mentioned earlier, the data signals may take any suitable form, depending on the required energy consumption, cost restrictions, and the degree of interference tolerated, etc. and may be, for example, an amplitude modulated signal, a frequency modulated signal, a pulse sequence with an SPL-depending frequency, etc. In the latter, for example, the microphone module circuit 8 may convert the microphone transducer 5 voltage to a pulse-train with a voltage-depending frequency. Each pulse briefly shorts the microphone module coil 31, 36, 37, 38, 91-96. This is sensed in the implant coil 30, 35, 41, 42, 60, 61, 62, 63, 65, 85-90 where a circuit converts the pulse-frequency back to a voltage representing the sound picked up by the microphone transducer 5. Hence, sound data is transferred wirelessly from the microphone module 1 to the implant, using power transmitted to the microphone module 1 by the implant.

In order to improve the energy efficiency of an inductive coupling, the microphone module 1 coil 31, 36, 37, 38, 91-96 is often tuned using a capacitor that causes it resonate at the frequency of the AC voltage. In PSK, the frequency to which the microphone module 1 coil 31, 36, 37, 38, 91-96 is tuned is modulated, which in turn, modulates the magnetic coupling field, and hence the implant inductive coil 30, 35, 41, 42, 60, 61, 62, 63, 65, 85-90 voltage. This can achieved by varying the capacitance of the tuning capacitor. Since varying the capacitance has a larger impact on the phase between the voltage across and the current through the inductive coil 30, 35, 41, 42, 60, 61, 62, 63, 65, 85-90 than on the amplitude of the coil voltage, the process is often referred to as PSK. It is an effect known in the art. The microphone module 1 in one embodiment of the invention transfers data signals to the implant in this manner, such data being, for example, sound received by the microphone transducer 5. As mentioned earlier, the data signals may take any suitable form, depending on the required energy consumption, cost restrictions, and the degree of interference tolerated, etc. and may be, for example, an amplitude modulated signal, a frequency modulated signal, a pulse sequence with an SPL-depending frequency, etc.

According to one aspect of the invention, the AC current generated by the implant inductive coil is modulated, such that the magnetic field generated and hence the voltage picked up by the microphone module coil is also modulated. By varying the modulation, the implant may transfer data signals to the microphone module, which information may be, for example, gain settings for a microphone amplifier.

### Conductive Coupling

According to another aspect of the invention, conductive coupling is used to send data signals from the implant to the microphone module 1 and/or vice versa. The implant interface 16 comprises one or more implant contacts which provide an electrical connection between the implant and the microphone module 2. According to one aspect of the invention the implant contacts are placed below the skin, meaning they are not in mechanical contact with the microphone module 2. This embodiment is depicted in FIG. 19 which shows a microphone module 1 of the invention, wherein the module interface 19 is a pair of electrical contacts 70, 71, and the implant interface 16 comprises a reciprocating pair of electrical contacts 73, 74 subcutaneously. Current is able to flow between the respective contacts owing to the conductivity of tissue. The current is preferably AC current, as a DC current is not suitable for use with the human body. Any net DC current, even a few μA/cm2, can lead over a period of time to irreversible electrolyte reactions that are toxic to the surrounding tissue (Robblee L. S. and T. L. Rose, “Electrochemical guidelines for selection of protocols and electrode materials for neural stimulation,” chapter 2 in Neural Prostheses—Fundamental Studies, Eds. Agnew W. F. and D. B McGreery, ISBN 0-13-615444-1, Prentice-Hall Inc., Englewood Cliffs, N.J. 07632, USA, p. 39, 1990). The AC current is again rectified to a DC voltage to supply the microphone circuitry 8 in a manner already described elsewhere herein. Data signals transfer information to the microphone module 1 by modulating the AC current. The microphone module 1 can transfer data signals information back to the implant by modulating its power consumption.

### Capacitive Coupling

According to another aspect of the invention, capacitive coupling is used to send data signals from the implant to the microphone module 1 and/or vice versa. A capacitive connection is formed by parallel-plate capacitors where one pair of plates inside and one pair of plates outside the body also passes AC current. The AC current is again rectified to a DC voltage to supply the microphone circuitry. The implant again transfers information to the microphone by modulating the AC current. The microphone also transfers audio or other information back to the implant by modulating its power consumption. In this embodiment, the respective interfaces comprise parallel-plate capacitors.

### Optical Coupling

According to another aspect of the invention, optical coupling is used to send data signals from the implant to the microphone module 1 and/or vice versa. An optical connection is formed by a light (e.g. infrared, visible) source in the implant interface 16 and a photovoltaic cell, in the module interface 19. The implant transfers information to the microphone module by modulating the source light output.

Data signals may be sent in the opposite direction, from the module to the implant using a light source in the module interface 19 and a photovoltaic cell in the implant interface 16. The microphone module transfers information to the implant by modulating the light output of its source, for example the intensity and/or wavelength. The photovoltaic cell is receptive to changes in intensity and/or wavelength. As mentioned earlier, the data signals may take any suitable form, depending on the required energy consumption, cost restrictions, and the degree of interference tolerated, etc. and may be, for example, an amplitude modulated signal, a frequency modulated signal, a pulse sequence with an SPL-depending frequency, etc.

### Transfer of Electrical Power

According to one aspect of the invention, the microphone module 1 receives electrical power from the implant. Thus, the microphone module 1 may be devoid of a self-contained power source, operating only when receiving electrical power from the implant. Alternatively, the microphone module 1 may have a rechargeable battery that is recharged using electrical power from the implant. The electrical power may be transferred from the implant to the microphone module 1 using magnetic (inductive) coupling, conductive coupling, non-conductive (i.e. capacitive) coupling or optical coupling. These modes of transfer have been described elsewhere herein, and are briefly discussed below.

### Inductive Coupling

Where electrical is power is transferred from the implant to the microphone module using magnetic (inductive) coupling, the implant interface 16 comprises an inductive coil that generates a magnetic flux using AC current, which flux is pickup up wirelessly by the module 1 interface 19 that comprise a reciprocating inductive coil 31, 36, 37, 38, 91-94 where an AC voltage is induced. Said AC voltage in the module 1 coil is rectified to a DC voltage to supply the microphone circuitry 8. Configurations of a power inductive coupling are shown in FIGS. 11, 12, 14 and 23 to 28 and explained elsewhere herein whereby the inductive coupling used to carry data signals is also used to transmit electrical to the microphone module 1.

According to one embodiment of the invention the implant interface 16 comprises separate coils to exchange data signals and to transfer electrical power. According to another embodiment of the invention, the microphone module 1 has separate coils to exchange data signals and to transfer electrical power. According to another aspect of the invention, the implant interface 16 and microphone module coils used to exchange data signals are the same as those used to transfer electrical power.

### Conductive Coupling

According to another aspect of the invention, conductive coupling is used to transfer electrical power from the implant to the microphone module 1. The implant interface 16 comprises one or more implant contacts which provide an electrical connection between the implant and the microphone module 2. According to one aspect of the invention the implant contact are placed below the skin, meaning they are not in mechanical contact with the microphone module 2. Current is able to flow between the contacts owing to the conductivity of tissue. The current is preferably AC current, as a DC current would cause the electrodes to release toxic metal ions into the surrounding tissue. The AC current is again rectified to a DC voltage to supply the microphone circuitry 8 in a manner already described elsewhere herein. Data signals can also be transfer information to the microphone module 1 by modulating the AC current. The microphone module 1 can also transfer data signals information back to the implant by modulating its power consumption.

According to one embodiment of the invention the implant interface 16 comprises separate contacts to exchange data signals and to transfer electrical power. According to another embodiment of the invention, the microphone module 1 has separate contacts to exchange data signals and to transfer electrical power. According to another aspect of the invention, the implant interface 16 and microphone contacts used to exchange data signals are the same as those used to transfer electrical power.

### Capacitive Coupling

According to another aspect of the invention, capacitive coupling is used to transfer electrical power from the implant to the microphone module 1. A capacitive connection may be formed by parallel-plate capacitors with one pair of plates below the skin and one pair of plates above the skin (i.e. in the microphone module) to pass AC current from the implant to the microphone module. The AC current is again rectified to a DC voltage to supply the microphone circuitry. The implant again transfers information to the microphone by modulating the AC current. The microphone also transfers audio information back to the implant by modulating its power consumption.

According to one embodiment of the invention the implant interface 16 comprises separate capacitors to exchange data signals and to transfer electrical power. According to another embodiment of the invention, the microphone module 1 has separate capacitors to exchange data signals and to transfer electrical power. According to another aspect of the invention, the implant interface 16 and microphone module 1 capacitors used to exchange data signals are the same as those used to transfer electrical power.

### Optical Coupling

According to another aspect of the invention, optical coupling is used to transfer power from the implant to the microphone module 1. An optical connection is formed by an energy providing light source in the implant interface 16 and a photovoltaic cell for the generation of electricity (e.g. solar-cell) in the module interface 19. The implant transfers power to the microphone module by emitting a light, preferably steady, through the tissue and skin onto the solar cell which converts the captured light flux into a DC current to power the microphone module circuitry.

According to one embodiment of the invention the implant interface 16 comprises separate light sources to exchange data signals and to transfer electrical power. According to another embodiment of the invention, the microphone module 1 has separate photovoltaic cells to exchange data signals and to transfer electrical power. According to another aspect of the invention, the implant interface 16 light sources used to exchange data signals are the same as those used to transfer electrical power. According to another aspect of the invention, microphone module 1 photovoltaic cells used to exchange data signals are the same as those used to transfer electrical power.

### Circuitry

The circuitry 8 present in the microphone module is operably connected to the microphone transducer 5 and interface 19 configured to receive electrical power from the interface 19, convert electrical signals generated by the microphone transducer 5 into data signals responsive to the electrical signals, and provide data signals to the interface 19.

In receiving electrical power from the interface 19, the circuitry 8 may comprise a rectifier for converting AC voltage to DC voltage when power is transferred using an inductive, conductive or capacitative coupling. When power is transferred using optical coupling, however, a rectification is not necessary. The circuitry may comprise a regulator for regulating the DC voltage to provide a constant output power source.

The circuitry 8 present in the microphone module 1 transforms the electrical signals from the microphone transducer 5 into data signals that are transferred by the module 1 interface 19 to the implant. To achieve transfer in the case of inductive, capacitive or conductive coupling, the circuitry 8 is configured to modulate the load current of the interface 19 responsive to the data signals; the load current is detected by the implant thereby transferring data signals via the interface 19 to the implant 101. In the case of an optical coupling for data transfer, the circuitry 8 is configured to change the intensity and/or wavelength of the light source responsive to the data signals; the change in light properties is detected by the implant thereby transferring data signals via the interface 19 to the implant 101.

The circuitry 8 present in the microphone module 1 may also be responsive to signals sent by the implant. To achieve this, circuitry 8 may be further configured to detect variations in voltage of electrical power received by the interface 19 from the implant 101, which variations correspond to data signals sent by the implant 101.

The circuitry 8 is electrically connected to the component discussed herein according to the practices of the person skilled in the art. The circuitry 8 comprises the necessary electronic components (e.g. wires, integrated circuits, switches etc) for converting current from the interface 19 to usable DC current for example, for converting AC current receive by induction from the interface 19 to usable DC current or regulating DC current generated by a photovoltaic cell in the interface 19. The circuitry 8 also comprises the necessary electronic components for performing the conversion of sound information into data signals, and for providing data signals to the interface 19, which components and configurations thereof are known in the art. An example of a wiring configuration is shown in FIG. 29 which illustrates a block diagram of both the microphone module 1 and implant 101. The microphone transducer 5 is wired 103 to the circuitry 8, electrical signals being transferred from the microphone in the direction of the arrow. The module interface 19 (e.g. coil, electrical contacts) is also wired 104, 104′ to the circuitry 8, electrical signals being transferred from the module interface 19 microphone in the direction of the arrows. As mentioned above, data transfer may be two way (107, 108); power may be transferred (107) from the implant 101 to the module 1. Data and/or power are passed without hardwiring (e.g. inductively, conductively, capacitively) across the skin 100. The implant 101 comprises a control unit 15 connected by wires 105, 105′, 106 to an implant interface 16 and an actuator 17. Typically an actuator may include one or more electrodes to electrically stimulate the auditory system, one or more vibration generators to mechanically stimulate the auditory system, or both.

As discussed already above, the module interface 19 may utilize an inductive, conductive capacitive and/or optical coupling, or any combination thereof.

Where an inductive coupling is employed, the circuitry 8 may rectify AC voltage induced in the module coil into DC, and regulate the voltage to provide a constant output power source.

The circuitry 8 may modulate the load current to allow data to be sent from the module 1 to the implant 101. This back-scattering effect is already discussed above. The circuitry 8 may also detect variations in the rectified DC voltage, allowing data signals to be transferred in the other direction, i.e. from the implant to the module 1, as already discussed above.

Where a conductive coupling is employed, the circuitry 8 may rectify AC current received by the module interface 19 to a DC voltage to supply the microphone circuitry 8 in a manner already described elsewhere herein. The circuitry 8 may modulate the load current to allow data to be sent from the module 1 to the implant 101. The circuitry 8 may detect modulations in the rectified DC voltage, allowing data signals to be transferred from the implant 101 to the module 1.

Where a capacitive coupling is employed, the circuitry 8 may rectify AC current flowing between the contacts to a DC voltage to supply the microphone circuitry 8 in a manner already described elsewhere herein. The circuitry 8 may modulate the load current to allow data to be sent from the module 1 to the implant 101. The circuitry 8 may detect modulations in the rectified DC voltage, allowing data signals to be transferred from the implant 101 to the module 1.

Where an optical coupling is employed, the circuitry 8 may regulate the voltage generated by the photovoltaic cell in the module interface 19 to provide an essentially constant output power source. The circuitry 8 may also detect variations in the generated DC voltage, allowing data signals to be transferred from the implant 101 to the module 1. A light source in the module interface 19 and reciprocating photovoltaic cell in the implant interface 16 may allow data transfer in the other direction, i.e. from the module 1 to the implant.

Besides the components to exchange data and power, the circuitry 8 may include other components such as a preamplifier, an analogue to digital converter, programmable memory, and a digital sound processor. It will provide the components needed to provide one or more of the signal types (e.g. amplitude modulation, frequency modulation etc.) mentioned above. It will provide the components needed to receive one or more of the signal types mentioned above. It may also include components to provide a telecoil functionality, light sensor functionality and radio receiver functionality for public room compatibility (see below).

According to one aspect of the invention, the circuitry 8 is programmable allowing its configuration to be set, for example using data signals from the implant. Parameters such as gain and sound-processing parameters can be changed depending on how the circuit 8 is programmed. The programming can be prepared to suit the patient's condition. The circuitry 8 may comprise a memory storage device for storing such programmable configurations. The circuitry 8 comprises the necessary electronic components (e.g. integrated circuits, memory chips, etc) for performing programmability, which components and configurations thereof are known in the art.

### Public Room Compatibility

The microphone module 1 may be provided with additional functionality allowing compatibility with public room hearing aids for the hard of hearing. According to one aspect of the invention, the microphone module 1 is provided with a telecoil to magnetically receive audio in telecoil-equipped rooms, from headsets, from telecoil-enabled telephones, or other compatible audio sources such as hi-fi, television, or alarm.

According to one aspect of the invention, the microphone module 1 is provided with a light sensor to receive audio in infrared-equipped rooms, infrared-enabled telephones, or other compatible audio sources such as hi-fi, television, or alarm. According to one aspect of the invention, the microphone module 1 is provided with a radio receiver to pick up audio in radio-equipped rooms, or other compatible audio sources such as hi-fi, television, or alarm. The module 1 and circuitry 8 will comprise the necessary electronic components (e.g. integrated circuits, antennas, sensors, etc) for performing additional functionality, which components and configurations thereof are known in the art.

### Other Features

According to one aspect of the invention the microphone module 1 may be provided with a withdrawal cord or pin to allow the patient to conveniently remove the module 1.

According to one aspect of the invention, the microphone module 1 comprises a coupling configured to engage with a placement tool, allowing insertion and removal of the module 1, preferably by the patient. The coupling provides a point of reversible attachment to the placement tool. The coupling may be provided as an opening, a protrusion, a ridge, a recess etc on the housing. Another aspect of the invention is a placement tool that grips the microphone module 1 during insertion and features a mechanical stop to prevent damage to the eardrum damage. The patient is able to safely insert the microphone module 1 without assistance from a specialist. The patient tool comprises a connecting means for engagement with the module 1 coupling means. It may be provided with a grip and release mechanism such as a push fitting, screw fitting, or similar.

### Double Use of the Implant Coil

Fully implantable middle-ear or cochlear implants have a battery that is recharged periodically through an inductive coupling, usually with an external coil that is held against the patient head, right on top of the implanted powering coil.

With such implants, it is possible to configure the implant coil that is normally used for battery recharge to also exchange data with and/or provide power to the microphone module. It requires that the microphone coil is tuned to the same frequency as the battery recharge circuit.

### Implant

The implant 101 comprising a control unit 15 an interface 16, and optionally an actuator 17, is an implant of the art adapted according to practices known to the skilled person to operate in conjunction with the transducer module 1, particularly the microphone module 1 described herein. A brief description of elements of an implant follows, relevant to a hearing implant. As mentioned elsewhere, the invention is not necessarily limited to a hearing implant. The skilled person will understand the implant can be adapted according to the type of transducer in the module. The actuator discussed below provides stimulation to the cochlea or an auditory nerve when the transducer is a microphone; however, when the transducer is, for instance, a camera, the actuator may provide stimulation to the optic nerve. Such adaptations are within the practices of the person skilled in the art.

### Actuator

An actuator 17 provides electrical stimulation to the cochlea or an auditory nerve, or to provide mechanical stimulation to the cochlea, or both. They are well known in the art. Where electrical stimulation is provided, the actuator will be a single, a pair and/or an array of electrodes. Where mechanical stimulation is provided, the actuator will be a vibration transducer e.g. electromagnetic, piezo-electric, electrostatic or magneto-restrictive transducer. The type of actuator will depend on the subject's condition, for example, whether residual hearing is present.

The actuator 17 is implantable. As such, it should fulfil the requirements for an implant such as biocompatible and stable housing, and be of suitable shape and size for insertion and placement. The parts of the actuator 17 in contact with tissue and/or body fluid may be made from any suitable biocompatible material. They may be made at least in part from or coated with, for example, surgical steels, or platinum, iridium, titanium, gold, silver, nickel, cobalt, tantalum, molybdenum, or their biocompatible alloys. They may, alternatively, be made from or coated with a biocompatible polymer such as PTFE or silicone polymer.

### Control Unit

A control unit 15 comprises circuitry to convert received data signal to corresponding electrical and/or mechanical stimulation, and to transmit electrical power using the implant interface 16. The control unit 15 comprises the necessary electronic components (e.g. integrated circuits, digital to analogue converts, digital signal processors, switches etc) for providing electrical power, which components and configurations thereof are known in the art.

The control unit 15 may be configured to perform some sound processing tasks. In one embodiment of the invention, the control unit 15 processes received sound information (data signals) and translates it into electrical signals carried to the electrodes, which are able to trigger nerves to fire neural signals (i.e. action potentials). Although the electrical signals are derived from sound, they do not resemble audio signals. Electrical signals may be, but not limited to, bursts of short bi-phasic pulses i.e. positive current pulse followed by an equal charge negative pulse. Typically, these pulses have a higher amplitude or a higher repetition rate when the sound information is louder. They are typically 10-100 μs long with μs edge transients, i.e. much shorter than audio signals. Such signals and processing thereto is known in the art, and the present method encompasses any processing tasks which convert sound information into signals suitable for stimulation of the auditory nerve.

In one embodiment of the invention, the control unit 15 processes received sound information and converts it into signals for sending to the vibration generator which in turn produces the corresponding mechanical vibrations to the inner-ear fluid or ossicles. The signal may be amplified.

In one embodiment of the invention, the control unit 15 is configured to send control signals using the data transfer methods mentioned above to the module 1. The control signals may adjust, for example, the gain or mode of operation. The control unit 15 may also be configured to receive information other than sound information, from the module 1, for example sensor data, mode data, gain settings etc, using also a data signal.

The control unit 15 may comprise a power source either directly housed in the unit, or electrically or magnetically connected thereto. The power source may be a disposable battery, preferably a long life battery (e.g. alkaline, lithium based). The power source may be a rechargeable battery (e.g. nickel cadmium, nickel metal hydride or lithium based). The battery may be recharged by externally accessible contacts, or by an induction coil.

The control unit 15 is implantable. As such, it should fulfil the requirements for an implant such as biocompatible and stable housing, and be of suitable shape and size for insertion and placement. The parts of the control unit 15 in contact with tissue and/or fluid may be made from any suitable biocompatible material. They may be made at least partly from of coated with, for example, surgical steels, or platinum, iridium, titanium, gold, silver, nickel, cobalt, tantalum, molybdenum, or their biocompatible alloys. They may, alternatively, be made from or coated with a biocompatible polymer such as PTFE or silicone polymer.

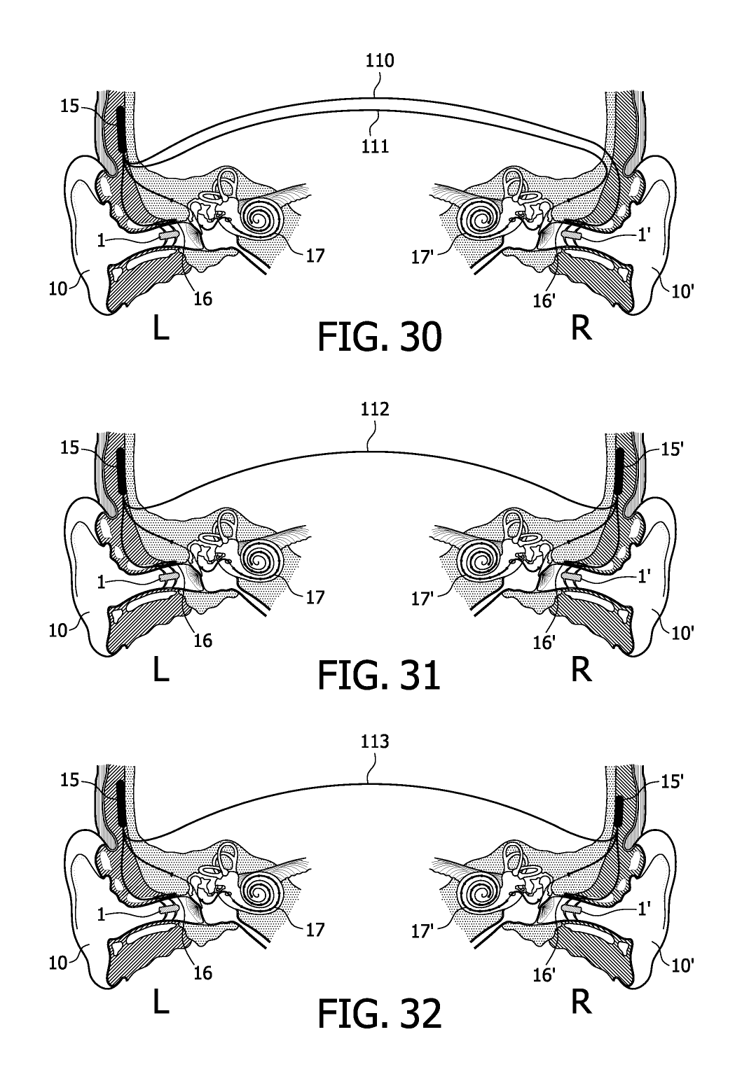

According to one embodiment of the invention, the control unit 15 provides a means to co-ordinate hearing from two microphone modules 1, one placed in each outer ear canal 12 or in each pinna 10. This can provide the subject with left-right directional sensitivity, particularly when microphones are placed in/on both ears. A single control 15 unit may control and process data received from two modules 1 and may send signals to two actuators 17 one implanted in each ear. An illustration of this embodiment is given in FIG. 30, showing a single left (L) implanted control unit 15 which provides signals to a left (R) 17 and right (R) 17′ actuator, and provides power to and exchanges data with a left 1 and right 1′ microphone module using a left 16 and right 16′ implant interface. The signal to the right actuator 17′ is transmitted across an electrical link 111 disposed subcutaneously between the left and right sides of the head. Data signals and power are exchanged across another electrical link 110 between the control unit 15 and the implant interface 16, which cable 110 is disposed subcutaneously between the left and right sides of the head.

Alternatively, two single control units 15 may perform this task of co-ordinate hearing from two microphone modules 1, communicating analog or digital information across a hardwired or wireless link. Each control unit 15 may be a fully contained independently powered implantable hearing aid, that uses audio information from both sides and produces the stimulation patterns for its associated ear. Furthermore, the control units 15 may exchange information on the settings of their processing algorithms to ensure balanced left-right stimulation. An illustration of this embodiment is given in FIG. 31, showing a left (L) 15 and right (R) 15′ implanted control unit, providing signals to a left (L) 17 and right (R) 17′ actuator respectively, and provides power to and exchanges data with a left 1 and right 1′ microphone module respectively, using a left 16 and right 16′ implant interface respectively. Coordinating data between the control units 15, 15′ is exchanged via either a single electrical or optical link 112 disposed subcutaneously between the left and right sides of the head, or via a wireless link.

Alternatively, one unit may operate as a master hearing aid, performing the signal processing for both ears, with the unit in the opposite ear merely acting as a slave unit relaying audio information to the master and converting stimulation information from the master into actuator signals for its associated ear. The slave unit may either have its own power source or obtain power from the master unit through the hardwired or wireless link. An illustration of this embodiment is given in FIG. 32, showing a left (L) 15 implanted master control unit and right (R) 15′ implanted slave control unit. The left (L) 15 master implanted control unit exchanges data with both the left 1 and right 1′ microphone module using a left 16 and right 16′ implant interface respectively. The left actuator receives signals directly from the left (L) 15 implanted master control unit, while the right (R) 15′ implanted slave control unit converts stimulation information from the master into actuator signals for its associated ear.

Implantable hearing aids typically contain a rechargeable battery that is recharged through an inductive link using an external coil that is held against the patient head. For patient convenience, the link between the two single control units 15 of any of the dual microphone configurations described above, may be configured to also transfer power from one side to the other to recharge the batteries. This allows recharging both control units simultaneously or consecutively with a single external coil that is held against the patient head.

### Interface

The implant 16 interface provides the means to transfer data signals between implant and module and/or to transfer power from the implant to the module by avoiding direct electrical contact between the implant and module. The implant 16 interfaces utilizes wireless means. As already elaborated elsewhere herein, preferably, the implant interface 16 uses a magnetic (inductive) coupling, a conductive coupling, a non-conductive (i.e. capacitive) coupling or an optical coupling. These four means for exchanging data signals and transferring electrical power are described in full above.

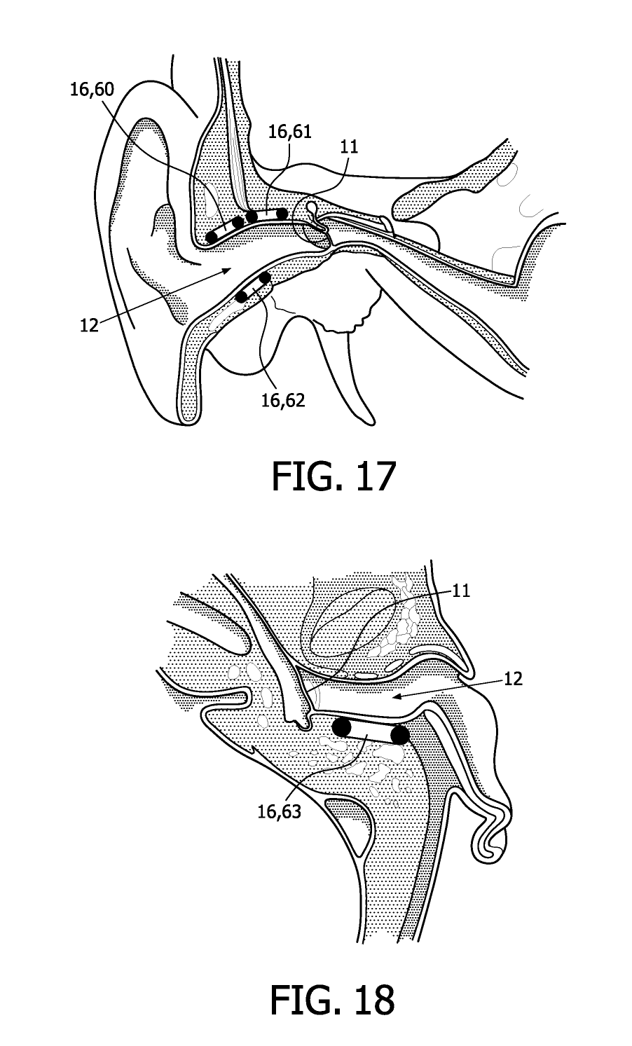

The implant interface 16 should be positioned cautiously during implantation to avoid fatigue failure of components therein particularly delicate interface wires. The skilled person will appreciate the close proximity of the mandibular joint (50, FIG. 16) to the external ear canal. The soft tissue of the ear canal moves substantially with the jaw movements and any wire loop or lead implanted in the vicinity 60, 62 of the soft tissue (FIG. 17) has a reduced life-span due to wire fatigue. Encapsulating the implant interface 16 in a rigid material, such as a medical grade epoxy or stiff silicone, and fixing it to the bony part of the ear canal (location 61, FIG. 17 or location 63, FIG. 18) solves this issue.

The implant interface 16 may be remote from the control unit 15; alternatively, it may be integrated in or on a housing of the control unit.

The implant interface 16 is implantable. As such, it should fulfil the requirements for an implant such as biocompatible and stable housing, and be of suitable shape and size for insertion and placement. The parts of the implant interface 16 in contact with tissue and/or fluid may be made at least partly from or coated with any suitable biocompatible material. These include surgical steel, or platinum, iridium, titanium, gold, silver, nickel, cobalt, tantalum, molybdenum, or their biocompatible alloys, Teflon®, PTFE, silicone polymer.

### Kit

One embodiment of the invention is a kit comprising a microphone module 1 as described above. The kit may further comprise an implant as described above. The kit may further comprise one or more replaceable protective membranes suitable for attachment to a sound port 4 of the module 1 as describe above. The kit may further comprise a protective membrane placement tool configured to allow attachment and/or removal the replaceable protective membrane from the module 1 as described above. The kit may further comprise a microphone module 1 placement tool, configured to allow a user to insert and/or remove the microphone module 1 from the outer ear canal 12 as described elsewhere herein.

1.-57. (canceled)

58. An externally worn transducer module (1) for use with an implant (101), which module (1) comprises:
a transducer (5) for converting energy into electrical signals,
a wireless interface (19), configured to transfer data signals to and/or from the implant (101), and receive electrical power from the implant (101),
circuitry (8) operably connected to the transducer (5) and interface (19) configured to:
receive electrical power from the interface (19),
convert electrical signals generated by the transducer into data signals responsive to the electrical signals, and
provide data signals to the interface (19), and
a housing (2) having a longitudinal axis (25) that forms a protective body of the module (1), which housing (1) is configured for external attachment to the body of the wearer.

59. The module (1) of claim 58, wherein the wireless interface (19) comprises at least one coil (31, 36—FIGS. 10, 11) for inductive coupling with the implant (101) orientated such that the axis of winding of the coil is essentially perpendicular or parallel to the longitudinal axis (25) of the housing (2).

60. The module (1) of claim 58, wherein the wireless interface (19) coil (31, 36—FIGS. 10, 11) is orientated such that the axis of winding of the coil is essentially perpendicular to the longitudinal axis (25) of the housing (2).

61. The module (1) of claim 58, wherein the wireless interface (19) coil (31, 36—FIGS. 10, 11) is orientated such that the axis of winding of the coil is essentially parallel to the longitudinal axis (25) of the housing (2).

62. The module (1) of claim 58, wherein the transducer is a microphone transducer (5), and the implant is a hearing implant (101).

63. The module (1) of claim 58, wherein the housing (1) is configured for insertion into the outer ear canal (12).

64. The module (1) of claim 58, wherein the housing (1) is configured for attachment to the pinna (10).

65. The module (1) of claim 58, wherein the circuitry (8) comprises a rectifier configured to convert AC voltage received by the wireless interface (19) to DC voltage, to provide said electrical power.

66. The module (1) of claim 58, wherein the circuitry (8) is configured to modulate the electric power consumption of the circuitry (8) responsive to the data signals, thereby transferring data signals via the interface (19) to the implant (101).

67. The module (1) of claim 58, wherein the circuitry (8) is configured to modulate the tuning frequency of the interface (19) responsive to the data signals, thereby transferring data signals via the interface (19) to the implant (101).

68. The module (1) of claim 58, wherein the circuitry (8) is further configured to detect variations in voltage of electrical power received by the interface (19) from the implant (101), which variations correspond to data signals sent by the implant (101).

69. The module (1) of claim 58, wherein the circuitry (8) is further configured to detect variations in frequency of electrical power received by the interface (19) from the implant (101), which variations correspond to data signals sent by the implant (101).

70. The module (1) of claim 58, wherein the circuitry (8) is further configured to detect variations in phase of electrical power received by the interface (19) from the implant (101), which variations correspond to data signals sent by the implant (101).

71. The module (1) of claim 58, whereby the data signals are amplitude modulated signals, frequency modulated signals, phase modulated signals, pulse width modulated signals, pulse sequences, pulse sequences with an SPL (sound pressure level)-depending frequency, pulse sequences with an SPL-depending pulse width, pulse sequences with an SPL-depending pulse phase, or a digitally encoded pulse sequences.

72. The module (1) of claim 62, whereby the circuitry (8) is configured to process the audio signal generated by the microphone transducer (5) prior to conversion into data signals.

73. The module (1) of claim 72, wherein said processing is dynamic compression or expansion.

74. The module (1) of claim 62, whereby the circuitry is configured to filter the audio signal generated by the microphone transducer (5) prior to conversion into data signals.

75. The module (1) of claim 74, whereby said filtering is low-pass, high-pass, or band-pass filtering.

76. The module (1) of claim 62, whereby the circuitry is configured to process the audio signal generated by the microphone transducer (5) to provide noise cancellation, frequency shifts, or suppression of Larsen feedback prior to conversion into data signals.

77. The module (1) of claim 58, further comprising a telecoil, operably connected to the circuitry (8).

78. The module (1) of claim 58, further comprising a light sensor, preferably an infrared sensor, operably connected to the circuitry (8).

79. The module (1) of claim 58, further comprising a radio receiver operably connected to the circuitry (8).

80. The module (1) of claim 62, where the housing comprises a sound port (4) configured to channel sound energy to the microphone transducer (5).

81. The module (1) of claim 80, where the sound port (4) comprises a means to receive a protective membrane.

82. The module (1) of claim 80, where the sound port (4) is covered by a protective membrane.

83. The module (1) of claim 81, wherein said protective membrane is replaceable.

84. The module (1) of claim 81, wherein said protective membrane is liquid impermeable, and gas or vapour permeable.

85. The module (1) of claim 81, wherein said protective membrane comprises Gore-tex.

86. The module (1) of claim 81, wherein said protective membrane comprises Gore-Tex XCR, event breathable fabric or Entrant breathable fabric.

87. The module (1) of claim 58, further comprising a withdrawal cord or pin.

88. The module (1) of claim 58, wherein at least part of the outer surface of the housing (2) is radially extended with one or more buffering structures (3, 3′), configured to bridge a gap between the inner wall of the outer ear canal (12) and the outer surface of the housing (2) in situ.

89. The module (1) of claim 88, wherein a buffering structure (3, 3′) is an annular ring, attached to the housing (2) towards a tympanic membrane end (7), and circumferentially extending outwards and backwards towards of the pinna end (6) of the housing (2), so creating a conical flap.

90. The module (1) of claim 89, wherein the extremity (28) of the buffering structure (3, 3′) describes a circle.

91. The module (1) of claim 89, wherein the extremity (28) of the buffering structure (3, 3′) describes an oval.

92. The module (1) of claim 88, wherein a buffering structure (3, 3′) is provided with one or more perforations (20) configured to allow the passage of sound waves therethrough.

93. The module (1) of claim 88, wherein a buffering structure (3, 3′) is provided with one or more notches (18) disposed towards the extremity (28) of the buffering structure (3, 3′).

94. The module (1) of claim 88, wherein the number of buffering structures (3, 3′) is between 1 and 4.

95. The module (1) of claim 88, wherein the buffering structure (3, 3′), is made of medical-grade silicone or rubber.

96. A kit comprising the transducer module (1) of claim 58.

97. The kit of claim 96, wherein the transducer is a microphone module.

98. The kit of claim 96, further comprising an implant having a control unit (15), and adapted to transfer data signals to and/or from the module (1), and to provide electrical power to the module (1) via an implant wireless interface (16) comprising at least one induction coil (30, 41, 42), which implant wireless interface (16) is operably connected to the control unit (15).

99. The kit of claim 98, wherein the implant wireless interface (16) comprises at least two induction coils (41, 42—FIGS. 12-13B) configured for implanting such that their respective axes of winding are essentially orthogonal to each other when the induction coil (36—FIG. 11) of the module (1); and wherein the wireless interface (19) coil (31, 36—FIGS. 10, 11) is orientated such that the axis of winding of the coil is essentially perpendicular to the longitudinal axis (25) of the housing (2).

100. The kit of claim 99, wherein the at least two induction coils (41, 42) are configured to generate a rotating field at the implant interface (16).

101. The kit of claim 98, wherein the implant wireless interface (16), comprises at least one induction coil (30) configured for implanting such that its axis of winding is essentially in parallel alignment with the longitudinal axis (25) of the module (1); wherein the wireless interface (19) coil (31, 36—FIGS. 10, 11) is orientated such that the axis of winding of the coil is essentially parallel to the longitudinal axis (25) of the housing (2).

102. The kit of claim 96, further comprising one or more replaceable protective membranes suitable for attachment to a sound port (4) of the module (1).

103. The kit of claim 102, where said protective membrane is replaceable; liquid impermeable and gas or vapour permeable; and/or comprises Gore-tex.

104. The kit of claim 103, wherein said replaceable protective membrane is a C-barrier.

105. The kit of claim 96, further comprising a protective membrane placement tool configured to allow attachment and/or removal a replaceable protective membrane from the module (1).

106. The kit of claim 96, further comprising a microphone module (1) placement tool, configured to allow a user to insert and/or remove the microphone module (1) from the outer ear canal (12).

107. An implant having a control unit (15), and adapted to transfer data signals to and/or from a module (1) of claim 58, and to provide electrical power to the module (1) via an implant wireless interface (16) comprising at least one induction coil (30, 41, 42), which implant wireless interface (16) implant wireless is operably connected to the control unit (15).

108. The implant of claim 107, wherein the implant wireless interface (16) comprises at least two induction coils (41, 42—FIGS. 12-13B) configured for implanting such that their axis of winding are essentially orthogonal to each other; and wherein the wireless interface (19) coil (31, 36—FIGS. 10, 11) is orientated such that the axis of winding of the coil is essentially perpendicular to the longitudinal axis (25) of the housing (2).

109. The implant of claim 108, wherein the at least two induction coils (41, 42) are configured to generate a rotating field at the implant interface (16).

110. The implant of claim 107, wherein the induction coil (30) is configured for implanting such that its axis of winding is essentially in parallel alignment parallel with the longitudinal axis (25) of the module (1); wherein the wireless interface (19) coil (31, 36—FIGS. 10, 11) is orientated such that the axis of winding of the coil is essentially parallel to the longitudinal axis (25) of the housing (2).
